(19) United States
(12) Patent Application Publication
(10) Pub. No.: US 2022/0117867 A1
(43) Pub. Date: Apr. 21, 2022

(54) ANTIMICROBIAL ACTIVITY OF FATTY ACID ESTERS AND COMBINATIONS THEREOF

(71) Applicant: SYMRISE AG, Holzminden (DE)

(72) Inventors: Christin KOCH, Detmold (DE); Steffen NORDZIEKE, Göttingen (DE); Jessica GRIEGER, Höxter (DE); Nikolas BUGDAHN, Holzminden (DE); Florian GENRICH, Dassel (DE); Sabine LANGE, Holzminden (DE)

(21) Appl. No.: 17/428,296

(22) PCT Filed: Jan. 22, 2020
(86) PCT No.: PCT/EP2020/051507

§ 371 Date: Aug. 4, 2021

(30) Foreign Application Priority Data
Feb. 4, 2019 (EP) PCT/EP2019/052576
Feb. 4, 2019 (EP) PCT/EP2019/052578
Feb. 4, 2019 (EP) PCT/EP2019/052582

(51) Int. Cl. A61K 8/37; A61Q 17/00; A61K 8/34

(52) U.S. Cl. CPC A61K 8/375; A61K 8/342; A61K 8/345; A61Q 17/005

(57) ABSTRACT

The present invention primarily relates to the use of a fatty acid ester, or a mixture comprising, or consisting of, two or more fatty acid esters, in a cosmetic product for inhibiting growth of microorganisms on a mammal's skin or mucosa, and/or preserving the cosmetic product against microbial growth, wherein the fatty acid ester, or at least one of the two or more fatty acid esters in the mixture, is selected from the group consisting of 3-hydroxypropyl caprylate, 3-hydroxypropyl undecylenate and glyceryl monoundecylenate.

The present invention primarily relates to a cosmetic product comprising or consisting of a fatty acid ester, or a mixture comprising or consisting of two or more fatty acid esters. Moreover, the present invention relates to various uses of the fatty acid ester, the mixture, or the cosmetic product.

Further aspects and embodiments of the present invention will arise from the description below, in particular from the examples, as well as from the appended patent claims.

The skin is a human body's largest organ. It is colonized by diverse microorganisms, most of which are harmless or even beneficial to their host. Colonization is driven by the ecology of the skin surface, which is highly variable depending on topographical location, endogenous host factors and exogenous environmental factors. For instance, the armpits present a rather humid and nutrient-rich habitat that provides favorable conditions for growth of many microorganisms. Other habitats include sebum-rich habitats like the face and rather dry and nutrient-poor habitats like most other body regions (Barnard & Li (2017) J Physiol 595.2 pp 437-450).

Many common skin pathogens, such as Staphylococcus aureus and Streptococcus pyogenes, are inhibited by an acidic pH, thus the growth of coagulase-negative staphylococci and corynebacteria is favored. However, skin occlusion results in an elevated pH, which favors the growth of S. aureus and S. pyogenes. Areas with a high density of sebaceous glands, such as the face, chest and back, encourage the growth of lipophilic microorganisms such as Propionibacterium spp and Malassezia spp.

Many skin disorders and infections can be caused by bacteria and fungi:

Chronic wounds, affecting diabetic, elderly, and immobile individuals, are an example where commensal skin organisms invade and become pathogenic upon breach of the skin barrier. Although bacteria do not cause the initial wounding event, they are thought to contribute to the lack of healing and persistent inflammation that is associated with chronic wounds. Burn wounds commonly become infected with S. pyogenes, Enterococcus spp. or Pseudomonas aeruginosa.

A further common skin commensal is Staphylococcus epidermidis. It is also the most frequent cause of hospital-acquired infection on in-dwelling medical devices such as catheters or heart valves.

Cosmetics, soaps, hygienic products and moisturizers are potential factors contributing to the variation of skin microbiota. These products alter the conditions of the skin barrier. However, their effects on skin microbiota remain unclear.

Hence, there is a need for products capable of controlling the skin microbiota, and in particular inhibit the growth of skin pathogens.

Cleansing and degerming the skin with a soap or detergent containing an antimicrobial agent may be a useful measure to prevent or treat skin disorders and infections. Short-chain and medium-chain fatty acids display good antimicrobial activity against some microorganisms, such as Malassezia, the causative pathogen of dandruff. However, their practical use in a cosmetic product for topical application is limited by their (i) intense smell, (ii) skin irritation, (iii) lack of skin-substantiveness and (iv) the difficulties they pose during formulation into products.

Various esters of said fatty acids do not display these disadvantageous properties. It is known that some fungi are able to cleave esters of said fatty acids and to release the fatty acids, leading to a “self-kill” of the fungi. However, the hydrolysis rates of esters through the microorganism's enzymes vary strongly depending on the alcohol component of the esters.

In DE 42 37 367 A1 fatty acid esters are described as antimycotic agents. These esters are preferably selected from the group of hexyl laurate, isopropyl stearate, glyceryl monolaurate, caprylic acid triglyceride and capric acid triglyceride.

DE 42 34 188 A1 relates to ethoxylated and propoxylated organic compounds as antimycotic agents in cosmetics.

DE 10 2004 046 603 A1 describes substance mixtures comprising fatty acid esters of polyols and salts of short chain fatty acids to counteract microorganisms.

SU 1286204 A1 discloses the use of a mixture of mono- (50-60%), di- (30-35%) and triesters (10-15%) of glycerol and undecylenic acid to give antimicrobial properties to a cosmetic base.

DE 33 14 786 A1 discloses a mixture with antimycotic activity comprising mono and/or di-10-undecylenic acid glyceryl esters. The mixtures are used in the treatment of nasal cavity mycosis and onychomycosis.

In WO 2006/054110 A2, esters of 1,2,3-propanetriol with one or more C11 to C24 fatty acids are described, wherein at least one fatty acid has at least one double bond. The application field for these substances is the treatment of chronic inflammatory disorders.

WO 2007/095262 A2 discloses 1,3-propanediol esters for the purpose of dissolving botanical extracts, fragrance concentrates and oils.

In view of the prior art, it is not known whether fatty acid esters have broad antimicrobial properties against a plurality of microorganisms of the natural microbiota of mammalian skin (herein also referred to in short as skin microorganisms), and/or or have microbiota balancing or reducing properties indirectly via sebum reduction. It is further not known whether fatty acid esters are suited for use in a cosmetic product, i.e. do not have a negative impact on physicochemical and/or sensory properties of the cosmetic product.

Unpublished, pending patent application PCT/EP2019/052576, filed on 4 Feb. 2019, discloses specific fatty acid esters and their use for treating an excess of Malassezia on the skin surface of mammals, the content of which is incorporated herein in its entirety.

Unpublished, pending patent application PCT/EP2019/052582, filed on 4 Feb. 2019, discloses specific fatty acid esters and their use in modifying the physicochemical properties, particularly increasing the viscosity or of the foam volume of a skin care product, the content of which is incorporated herein in its entirety.

Unpublished, pending patent application PCT/EP2019/052578, filed on 4 Feb. 2019, discloses specific fatty acid esters and their use in modifying the sensory properties of a skin care product, particularly improving the foam formation on the skin or of the skin sensation, the content of which is incorporated herein in its entirety.

It has now been further found by the inventors of the present application that the therein disclosed fatty acids exhibits activity against further microorganisms, particularly bacteria and fungi of the mammalian natural microbiota of the skin or mucosa.

It was thus an object of the present invention to provide active agents that are effective against a variety of microorganisms, in particular bacteria and/or fungi, of the natural microbiota of mammalian skin or mucosa, while at the same time meet an ingredient's requirements for inclusion into a cosmetic product for topical application. In particular, the active agents should not have an intense smell, should not cause skin irritation, should have good skin-substantiveness and should be easy to formulate. Moreover, the active agents should have a neutral, preferably positive, impact on physicochemical and/or sensory properties of the cosmetic product.

According to a first aspect of the present invention, the object is achieved by a cosmetic product comprising:

a fatty acid ester, or

a mixture comprising, or consisting of, two or more fatty acid esters,

wherein the fatty acid ester, or at least one of the two or more fatty acid esters in the mixture, are selected from the group consisting of:

3-hydroxypropyl caprylate, 3-hydroxypropyl undecylenate and glyceryl monoundecylenate, preferably from the group consisting of 3-hydroxypropyl caprylate, and 3-hydroxypropyl undecylenate, and

more preferably, wherein the fatty acid ester, or one of the fatty acid esters, is 3-hydroxypropyl caprylate.

As used herein, the term “comprising” means that the named elements are essential, but other elements may be added and is still embraced by the present invention, whereas the term “consisting of” signifies that the subject matter is closed-ended and can only include the limitations that are expressly recited. Whenever reference is made to “comprising” it is intended to cover both meanings as alternatives, that is the meaning can be either “comprising” or “consisting of”, unless the context dictates otherwise.

The present invention is based on the inventors' innovation that specific fatty acid esters confer an advantageous property profile to a cosmetic product, which has not been achieved before. First, the specific fatty acid esters have been found to exhibit antimicrobial activity against a plurality of microorganisms, in particular bacteria and/or fungi, of the natural microbiota of mammalian skin or mucosa. The cosmetic product can therefore be usefully employed in controlling the natural microbiota of the skin or mucosa. Second, the specific fatty acid esters have been found to exhibit antimicrobial activity against microorganisms that are commonly used for preservative testing. Therefore, the cosmetic product is expected to have a good shelf-life. Third, the specific fatty acid esters are associated with improved physicochemical and sensory properties. Addition of the specific fatty acid esters to a cosmetic product thus results in improved physicochemical and sensory properties of the cosmetic product, in particular in terms of an increase of the viscosity, an increase of the foam volume, an improvement of the foam formation on the skin, and an improvement of the skin sensation. In addition, it has been found that the specific fatty acid esters disclosed herein have microbiota balancing or reducing properties indirectly via sebum reduction. The specific fatty acid esters as disclosed herein are therefore a universal tool for conferring beneficial properties to a cosmetic product and can be advantageously used for different types of cosmetic products including crèmes, lotions, shampoos, etc. (further examples are given below).

The cosmetic product as disclosed herein comprises at least one of a specified fatty acid ester. Moreover, it is to be understood that the at least one specified fatty acid ester is present in an antimicrobial effective amount. An antimicrobial effective amount of a compound is an amount, which has to be added to a cosmetic product in order to observe an antimicrobial effect associated with said compound. In preferred cases, the observed antimicrobial effect may be due to a cooperation (e.g. synergism) of the at least one specified fatty acid ester with a further compound. To determine the antimicrobial effective amount of the at least one specified fatty acid ester in a cosmetic product sample, like samples may be prepared that contain different amounts of the at least one specified fatty acid ester, and at least a further like sample that does not contain the at least one specified fatty acid ester. These samples may be used in different antimicrobial tests as described in the examples section herein.

An increase of the viscosity, an increase of the foam volume, an improvement of the foam formation on the skin, and an improvement of the skin sensation can be determined by following routine methods or in analogy to PCT/EP2019/052582, and PCT/EP2019/052578.

As non-limiting examples of mixtures comprising two or more fatty acid esters, mixtures that comprise or consist of one, two, three or all of the two or more fatty acid esters in the mixture are mentioned.

Caprylate refers to an ester of caprylic acid (CAS Registry Number of caprylic acid: 124-07-2; also known as octanoic acid) and undecylenate refers to an ester of 10-undecylenic acid (CAS Registry Number of 10-undecylenic acid: 112-38-9; also known as 10-undecenoic acid).

3-Hydroxypropyl caprylate refers to the monoester of the alcohol 1,3-propanediol (CAS Registry Number: 504-63-2) with caprylic acid, and 3-hydroxypropyl undecylenate refers to the monoester of the alcohol 1,3-propanediol with 10-undecylenic acid.

A preferred embodiment relates to a cosmetic product (as disclosed herein), wherein the cosmetic product is a cosmetic product for topical application on a mammal's skin or mucosa. The mammal is preferably a human. For topical application, the cosmetic products are either rinse off or leave on preparations preferably taking the form of a cleanser, an o/w or w/o emulsion, serum, scrub, lotion, soap, ointment, wax, powder, solution, emulsion, paste, suspension, tablet, gel, stick, aerosol, most preferably taking the form of an aerosol-based deo spray, deo pump spray, deo stick, deo roll on, deo cream, deo wipes, deo crystals, pickering emulsions, hydrodisperion gels, skin balms, shampoo, shower gel, foam bath, micellar water, facial cleansing solutions, cleansing wipes, intimate spray, intimate cream, intimate wash lotion, intimate wipes, aerosol-based foot spray, foot pump spray, foot bath, foot balm, mouth wash concentrate, mouth wash ready to use, and tooth paste.

According to a further preferred embodiment, the cosmetic product is selected from the group consisting of body (including foot) deodorant care products (for example deo spray aerosol based and deo pump spray, deo stick, deo roll on, deo cream, deo wipes, deo crystals, water free deo products), skin care (for example o/w emulsions, w/o emulsions, multiple emulsions, pickering emulsions, hydrodisperion gels, balms, water free products) & cleansing products (for example shampoo, shower gel, foam bath, micellar water, facial cleansing solutions, water free cleansing products, cleansing wipes), intimate care & cleansing products (for example spray, cream, lotion, fluid, surfactant based rinse off preparations, wipes), foot care & cleansing products (for example aerosol spray, pump spray, foot bath, o/w & w/o emulsion, balm), and oral care products (for example mouth wash concentrate, mouth wash ready to use, tooth paste). That is, the cosmetic product is primarily intended to be applied under the armpit, to the face, to the feet, to the intimate region or to the oral mucosa for the primary purposes of providing a hygienic and “healthy” feeling. It is advantageous to formulate the cosmetic product as disclosed herein in a way that enables users to incorporate the desired effects, in particular the desired antimicrobial effect, into their daily hygienic routine. This is achieved by, for example, providing the cosmetic product in the form of the above-mentioned (daily) care products.

A further embodiment of the invention relates to a cosmetic product as defined herein comprising 3-hydroxypropyl caprylate or 3-hydroxypropyl undecylenate or glyceryl monoundecylenate.

Another embodiment of the invention relates to a cosmetic product as defined herein comprising a mixture comprising:

3-hydroxypropyl caprylate or 3-hydroxypropyl undecylenate or glyceryl monoundecylenate, and

at least one further fatty acid ester that does not belong to the fatty acid esters specified in claim 1 (herein also referred as the specified fatty acid esters).

Another embodiment of the invention relates to a cosmetic product as defined herein comprising a mixture comprising at least two fatty acid esters, wherein at least one fatty acid ester is selected from the group consisting of:

3-hydroxypropyl caprylate, 3-hydroxypropyl undecylenate and glyceryl monoundecylenate, and at least one further fatty acid ester is selected from the group consisting of:

3-hydroxypropyl caprylate, glyceryl monocaprylate, 3-hydroxypropyl undecylenate and glyceryl monoundecylenate.

Another preferred embodiment of the invention relates to a cosmetic product as defined herein, further comprising one or more 1,2-alkane diol(s), preferably one or more 1,2-alkane diol(s) selected from the group consisting of:

(i) 1,2-pentanediol, 1,2-hexanediol, 1,2-octanediol and 1,2-decanediol, more preferably one 1,2-alkane diol selected from the group consisting of 1,2-pentanediol, 1,2-hexanediol, 1,2-octanediol and 1,2-decanediol, in particular 1,2-octanediol, and/or

(ii) one or more compounds selected from the group consisting of dimethyl phenylbutanol, glyceryl caprylate and Farnesol.

Surprisingly, it was found that the addition of one or more 1,2-alkane diol(s) as defined herein, or a compound as described in item (ii) above, to one or more fatty acid ester as defined herein leads to a synergistic increase in antimicrobial activity against a plurality of microorganisms (as will be demonstrated further below). In terms of a synergistic effect, it is particularly preferred that the one or more fatty acid ester is/are caprylic acid ester(s) or undecylenic acid ester(s) as defined herein, preferably 3-hydroxypropyl caprylate.

Thus, a preferred embodiment of the invention relates to a cosmetic product comprising:
1,2-pentanediol and 3-hydroxypropyl caprylate, or
1,2-pentanediol and 3-hydroxypropyl undecylenate, or
1,2-hexanediol and 3-hydroxypropyl caprylate, or
1,2-hexanediol and 3-hydroxypropyl undecylenate, or
1,2-octanediol and 3-hydroxypropyl caprylate, or
1,2-octanediol and 3-hydroxypropyl undecylenate, or
1,2-decanediol and 3-hydroxypropyl caprylate, or
1,2-decanediol and 3-hydroxypropyl undecylenate, or
1,2-pentanediol and glyceryl monoundecylenate, or
1,2-hexanediol and glyceryl monoundecylenate, or
1,2-octanediol and glyceryl monoundecylenate, or
1,2-decanediol and glyceryl monoundecylenate.

Another embodiment of the invention relates to a cosmetic product as defined herein comprising a mixture of:
3-hydroxypropyl caprylate and 3-hydroxypropyl undecylenate, or
glyceryl monocaprylate and 3-hydroxypropyl undecylenate, or
3-hydroxypropyl caprylate and glyceryl monoundecylenate, or
glyceryl monocaprylate and glyceryl monoundecylenate, or
3-hydroxypropyl caprylate and glyceryl monocaprylate, or
3-hydroxypropyl caprylate and 3-hydroxypropyl undecylenate, or
glyceryl monocaprylate and 3-hydroxypropyl undecylenate, or
3-hydroxypropyl caprylate and glyceryl monoundecylenate, or
glyceryl monocaprylate and glyceryl monoundecylenate, or
3-hydroxypropyl caprylate and glyceryl monocaprylate, preferably
3-hydroxypropyl caprylate and glyceryl monocaprylate.

Another embodiment of the invention relates to a cosmetic product as defined herein comprising a mixture of:
3-hydroxypropyl caprylate, glyceryl monocaprylate and 3-hydroxypropyl undecylenate, or
3-hydroxypropyl caprylate, glyceryl monocaprylate and 3-hydroxypropyl undecylenate.

Another embodiment of the invention relates to a cosmetic product comprising a mixture of:
3-hydroxypropyl caprylate, glyceryl monocaprylate, 3-hydroxypropyl undecylenate and glyceryl monoundecylenate, or
3-hydroxypropyl caprylate, glyceryl monocaprylate, 3-hydroxypropyl undecylenate and glyceryl monoundecylenate.

As described above, the cosmetic product according to the various embodiments described herein may further comprise one or more 1,2-alkane diol(s) as defined herein.

In some embodiment, there may be at least one further fatty acid ester that does not belong to the specified fatty acid esters. In other embodiments, the specified fatty acid ester(s) is (are) the only fatty acid esters comprised in the cosmetic product.

Another preferred embodiment of the invention relates to a cosmetic product as defined herein, wherein the cosmetic product does not comprise any diesters of caprylic acid and/or does not comprise any triesters of caprylic acid and/or does not comprise any ethoxylated variants of caprylic acid and/or does not comprise any diesters of undecylenic acid and/or does not comprise any triesters of undecylenic acid and/or does not comprise any ethoxylated variants of undecylenic acid.

Advantageously, the specified fatty acid esters show particularly high antimicrobial activity against a variety of microorganisms of the natural microbiota of mammalian skin and mucosa. Particularly, binary mixtures of fatty acid esters display a synergistic increase in antimicrobial activity (as will be demonstrated further below). Of particular value in this respect are binary mixtures of a fatty acid ester as disclosed herein and at least one of an 1,2-alkanediol, dimethyl phenylbutanol, glyceryl caprylate and Farnesol. Preferably, in the binary mixtures, the fatty acid ester is 3-hydroxypropyl caprylate. Further preferred is that the 1,2-alkanediol, if present in a binary mixture as disclosed herein, is 1,2-pentanediol, 1-2, hexanediol, or 1,2-octanediol, preferably 1,2 octanediol.

Another preferred embodiment of the present invention relates to a cosmetic product as defined herein, comprising one or more additional active agent(s), preferably one or more antimicrobial agent(s), more preferably one or more active agent(s) selected from the group consisting of clotrimazole (CAS Registry Number 23593-75-1), bifonazole (CAS Registry Number 60628-96-8), miconazole (CAS Registry Number 22916-47-8), ketoconazole (CAS Registry Number 65277-42-1), fluconazole (CAS Registry Number 86386-73-4), climbazole (CAS Registry Number 38083-17-9), itraconazole (CAS Registry Number 84625-61-6), terbinafine (CAS Registry Number 91161-71-6), nystatin (CAS Registry Number 1400-61-9), amorolfine (CAS Registry Number 78613-35-1), ciclopirox (CAS Registry Number 29342-05-0), octopirox (CAS Registry Number 68890-66-4) and undecylenic acid (CAS Registry Number 112-38-9).

A preferred alternative embodiment relates to a cosmetic product as defined herein, which is used in combination with one or more additional active agent(s), preferably with one or more antimicrobial agent(s), more preferably with one or more active agent(s) selected from the group consisting of 2-Methyl 5-cyclohexylpentanol, Dimethyl phenylbutanol, Di methyl phenylpropanol, 2-benzylheptanol, Ethylhexylglycerin, Triethyl citrate, Polyglyceryl-3 caprylate, Polyglyceryl-10 Laurate, glyceryl caprate, polyglyceryl-2 caprate, Zinc ricinoleate, Farnesol, bisabolol, Glyceryl laurate, glyceryl caprylate, Xylityl Sesquicaprylate, decylene glycol, caprylyl glycol, phenylpropanol, lauryl alcohol, o-cymen-5-ol, Octenidine HCl, Undecylenoyl Glycine, Sodium Caproyl/Lauroyl Lactylate, Ethyl Lauroyl, Arginate Laurate, PCA, Ethyl Cocoyl Arginate, Salvia officinalis (Sage) Oil, tropolone, piroctone olamine, triclosan, triclocarban, chloroxylenol, chlorocresol, benzalkonium chloride, benzalkonium bromide, benzalkonium saccharinate, benzethonium chloride, chlorphenesin, alkyl (C12-22) trimethyl ammonium bromide and chloride, zinc pyrithione, climbazole, chlorhexidine, chlorhexidine diacetate, chlorhexidine digluconate, chlorhexidine dihydrochloride, hexamidine, hexamidine diisethionate, silver chloride, and silver citrate.

According to a preferred embodiment, the total amount of the fatty acid ester(s) (as defined herein) comprised in the cosmetic product as defined herein is from 0.01 to 5 wt.-%, more preferably from 0.05 to 2 wt.-%, most preferably from 0.1 to 1 wt.-%, relative to the total weight of said product.

According to another preferred embodiment, in cosmetic products comprising a mixture of two fatty acid esters, the weight ratio between the two fatty acid esters as defined herein is from 10:1 to 1:10, more preferably from 5:1 to 1:5 and most preferably from 3:1 to 1:3.

According to a preferred embodiment, the total amount of the fatty acid ester(s) (as defined herein) and 1,2-alkane diol(s) (as defined herein) comprised in the cosmetic product as defined herein is from 0.1 to 10 wt.-%, more preferably from 0.2 to 5 wt.-%, most preferably from 0.3 to 3 wt.-%, relative to the total weight of said product.

According to another preferred embodiment, the weight ratio between the fatty acid ester(s) (as defined herein) and the 1,2-alkane diol(s) (as defined herein) comprised in the cosmetic product as defined herein is from 20:1 to 1:20, more preferably from 10:1 to 1:10 and most preferably from 5:1 to 1:5.

A second aspect of the present invention relates to the use of a fatty acid ester, or a mixture comprising two or more fatty acid esters, in a cosmetic product for:

(i) inhibiting growth of microorganisms on a mammal's skin or mucosa; and/or

(ii) preserving the cosmetic product, and

optionally:

(iii) modifying physicochemical properties of the cosmetic product, and/or

(iv) improving sensory properties of the cosmetic product on a mammal's skin or mucosa, and/or

(v) balancing and/or reducing sebum production,

wherein the fatty acid ester, or at least one of the two or more fatty acid esters in the mixture, are selected from the group consisting of:

3-hydroxypropyl caprylate, 3-hydroxypropyl undecylenate and glyceryl monoundecylenate, preferably from the group consisting of 3-hydroxypropyl caprylate, and 3-hydroxypropyl undecylenate, and

more preferably, wherein the fatty acid ester, or one of the fatty acid esters, is 3-hydroxypropyl caprylate.

The fatty acid ester(s) and the cosmetic product of the second aspect are identical to the fatty acid ester(s) and the cosmetic product of the first aspect. That is, the various embodiments described in the context of the first aspect form corresponding embodiments of the second aspect.

According to a preferred embodiment, the use of a fatty acid ester, or a mixture comprising two or more fatty acid esters, in a cosmetic product as disclosed herein is for:

(i) inhibiting growth of microorganisms on a mammal's skin or mucosa; and

(ii) optionally preserving the cosmetic product; and

(iii) optionally modifying physicochemical properties of the cosmetic product; and

(iv) optionally improving sensory properties of the cosmetic product on a mammal's skin or mucosa; and

(v) optionally balancing and/or reducing sebum production.

According to a further preferred embodiment, the use of a fatty acid ester, or a mixture comprising two or more fatty acid esters, in a cosmetic product as disclosed herein is for:

(i) inhibiting growth of microorganismson a mammal's skin or mucosa; and

(ii) optionally preserving the cosmetic product; and

(iii) modifying physicochemical properties of the cosmetic product, and

(iv) optionally improving sensory properties of the cosmetic product on a mammal's skin or mucosa; and

(v) optionally balancing and/or reducing sebum production.

According to a further preferred embodiment, the use of a fatty acid ester, or a mixture comprising two or more fatty acid esters, in a cosmetic product as disclosed herein is for:

(i) inhibiting growth of microorganisms on a mammal's skin or mucosa; and

(ii) optionally preserving the cosmetic product; and

(iii) optionally modifying physicochemical properties of the cosmetic product, and

(iv) improving sensory properties of the cosmetic product on a mammal's skin or mucosa; and

(v) optionally balancing and/or reducing sebum production.

According to a further preferred embodiment, the use of a fatty acid ester, or a mixture comprising two or more fatty acid esters, in a cosmetic product as disclosed herein is for:

(i) inhibiting growth of microorganisms on a mammal's skin or mucosa; and

(ii) optionally preserving the cosmetic product; and

(iii) modifying physicochemical properties of the cosmetic product, and

(iv) improving sensory properties of the cosmetic product on a mammal's skin or mucosa; and

(v) optionally balancing and/or reducing sebum production.

According to a further preferred embodiment, the use of a fatty acid ester, or a mixture comprising two or more fatty acid esters, in a cosmetic product as disclosed herein is for:

(i) inhibiting growth of microorganisms on a mammal's skin or mucosa; and

(ii) preserving the cosmetic product; and

(iii) modifying physicochemical properties of the cosmetic product, and

(iv) improving sensory properties of the cosmetic product on a mammal's skin or mucosa; and

(v) optionally balancing and/or reducing sebum production.

According to a further preferred embodiment, the use of a fatty acid ester, or a mixture comprising two or more fatty acid esters, in a cosmetic product as disclosed herein is for:

(i) inhibiting growth of microorganisms on a mammal's skin or mucosa; and

(ii) preserving the cosmetic product; and

(iii) modifying physicochemical properties of the cosmetic product, and

(iv) improving sensory properties of the cosmetic product on a mammal's skin or mucosa; and

(v) balancing and/or reducing sebum production.

Preferably, the use is a cosmetic use and/or a non-therapeutic use. This means, it is desirable to use the fatty acid ester or mixture as defined herein cosmetically/non-therapeutically to inhibit the growth of microorganisms on the mammal's skin or mucosa, in particular before a condition or infection of the skin develops, or before any disease symptoms can be observed. It is, for example, possible to use the fatty acid ester(s) or mixtures as defined herein to improve unpleasant conditions associated with excessive sebum production, redness, itching, dryness, flaking, greasiness, hypopigmentation and/or hyperpigmentation of the skin or mucosa, which would not (yet) be categorized as a disease condition.

A preferred embodiment relates to a use, wherein the inhibition of the growth of microorganisms is a reduction of the microorganisms' growth rate, and/or a stagnation or reduction of the number of living microorganisms. A further preferred embodiment of the invention relates to a use, wherein the modification of the sensory properties is an improvement of the foam formation on the skin or mucosa, and/or of the skin or mucosa sensation of the cosmetic product. A further preferred embodiment of the invention relates to a use, wherein the modification of the physicochemical properties is an increase of the viscosity and/or of the foam volume of the cosmetic product.

A use is further preferred, wherein the microorganismsare selected from the group consisting of microorganisms, in particular bacteria and/or fungi, of the natural microbiota of mammalian skin or mucosa. Preferably, the microorganismsare selected from the group consisting of microorganisms from the genus; Staphylococcus; Corynebacterium; Aspergillus; Candida; Escherichia; Peptoniphilus; Streptococcus; Lactobacillus; Gardnerella; Fannyhessea; Epidermophyton; Trichophyton; Fusobacterium, and combinations threreof. Preferably, the microorganisms are selected from the group consisting of: S. epidermidis; Staphylococcus hominis; Corynebacterium xerosis; Aspergillus brasiliensis; Candida albicans; Escherichia coli; Staphylococcus aureus; Peptoniphilus lacrimalis; Streptococcus agalactiae; Lactobacillus acidophilus; Gardnerella vaginalis; Fannyhessea vaginae (Atopobium vaginae); Epidermophyton floccosum; Trichophyton rubrum; Streptococcus mutans; Fusobacterium nucleatum, and combinations threreof. The cosmetic product as disclosed herein showed excellent antimicrobial activities against these microorganisms.

A third aspect of the present invention relates to a cosmetic product as disclosed herein for use in a method of antimicrobial therapy of a mammal by topical application of the cosmetic product on the mammal's skin or mucosa in at least a region of the skin or mucosa infested with microorganisms. Again, the various embodiments described in the context of the first aspect form corresponding embodiments of the third aspect.

For the purposes of the present invention, the term “therapy” encompasses prevention of a condition of the skin or mucosa, or a prevention of symptoms associated with said condition, or a restoral of the health of a mammal having a condition of the skin or mucosa, or relief of symptoms associated with said condition. Prevention can be achieved by applying the cosmetic product as defined herein in repeated time intervals such as once in a week, once every other day, or once daily, to regions of the skin or mucosa, where occurrence of a condition should be avoided. Treatment can be achieved by applying the cosmetic product once or repeatedly to a region of the skin or mucosa, which shows symptoms of a condition of the skin or mucosa.

According to a preferred embodiment, the antimicrobial therapy is a therapy of a condition or infection of the skin or mucosa involving an excess of the microorganisms in at least the region of the skin or mucosa. As defined herein, an excess of microorganisms on the skin or mucosa of a mammal, preferably a human, relates to a situation where the total amount of cells of a given microbial species present on said skin or mucosa leads to symptoms of conditions or infections of the skin or mucosa such as, for example, redness, itching, dryness, flaking, greasiness, hypopigmentation and/or hyperpigmentation of the skin.

According to a further preferred embodiment, the microorganisms are selected from the group consisting of microorganisms, in particular bacteria and/or fungi, of the natural microbiota of mammalian skin or mucosa. Preferably, the microorganisms are selected from the group consisting of microorganisms from the genus Staphylococcus; Corynebacterium; Aspergillus; Candida; Escherichia; Peptoniphilus; Streptococcus; Lactobacillus; Gardnerella; Fannyhessea; Epidermophyton; Trichophyton; Fusobacterium, and combinations threreof. Preferably, the microorganisms are selected from the group consisting of: S. epidermidis; Staphylococcus hominis; Corynebacterium xerosis; Aspergillus brasiliensis; Candida albicans; Escherichia coli; Staphylococcus aureus; Peptoniphilus lacrimalis; Streptococcus agalactiae; Lactobacillus acidophilus; Gardnerella vaginalis; Fannyhessea vaginae (Atopobium vaginae); Epidermophyton floccosum; Trichophyton rubrum; Streptococcus mutans; Fusobacterium nucleatum, and combinations threreof.

In some embodiments, the microorganisms do not belong to the genus of Malassezia.

A method for reducing the growth of microorganisms on the skin or mucosa of mammals, preferably humans, in particular of humans in need thereof (i.e. of humans showing one or more symptoms of a (unpleasant or disease) condition of the skin or mucosa (such as, for example, body or scalp malodor, redness, itching, dryness, flaking, greasiness, hypopigmentation and/or hyperpigmentation of the skin) comprising the step of applying a cosmetic product as disclosed herein to a region of the skin or mucosa, is further disclosed herein. The cosmetic product is preferably selected from the group consisting of body (including foot) deodorant care products (for example deo spray aerosol based and deo pump spray, deo stick, deo roll on, deo cream, deo wipes, deo crystals, water free deo products), skin care (for example o/w emulsions, w/o emulsions, multiple emulsions, pickering emulsions, hydrodisperion gels, balms, water free products), cleansing products (for example shampoo, shower gel, foam bath, micellar water, facial cleansing solutions, water free cleansing products, cleansing wipes), intimate care & cleansing products (for example spray, cream, lotion, fluid, surfactant based rinse off preparations, wipes) foot care & cleansing products (for example aerosol spray, pump spray, foot bath, o/w & w/o emulsion, balm) and oral care products (for example mouth wash concentrate, mouth wash ready to use, tooth paste). Accordingly, the method as disclosed herein is particularly useful for the purpose of improving body odor, hygiene and/or cleansing.

The present disclosure also relates to a cosmetic product as disclosed herein, wherein the fatty acid ester is glyceryl monocaprylate in place of 3-hydroxypropyl caprylate, 3-hydroxypropyl undecylenate and/or glyceryl monoundecylenate.

The various aspects and embodiments described herein are meant to be combinable with each other unless the context dictates otherwise.

In the following drawings:

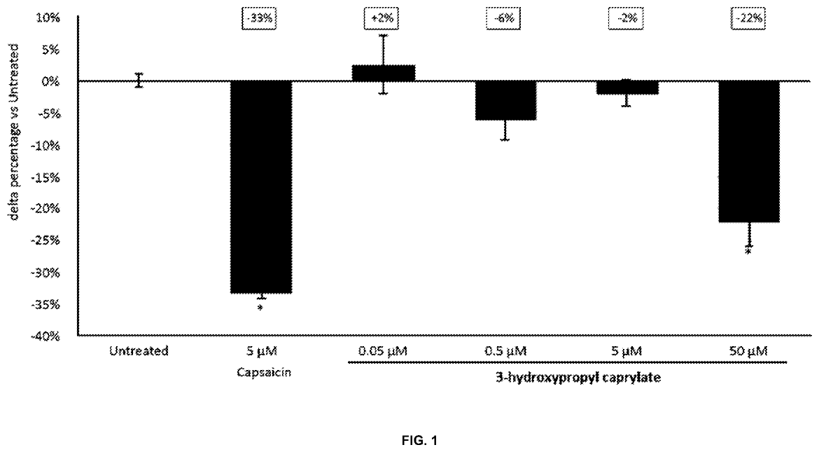

FIG. 1 show the relative change in total lipids in human sebaceous glands of Donor 1. Asterisk indicates a significant difference compared to untreated control;

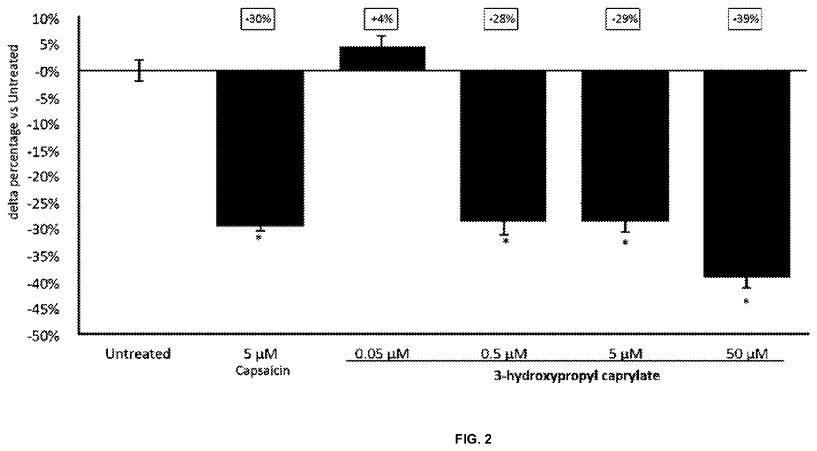

FIG. 2 show the relative change in total lipids in human sebaceous glands of Donor 2. Asterisk indicates significant difference compared to untreated control.

The invention will now be described in more detail hereinafter with references to selected examples.

### EXAMPLES

1. Methods

A) Minimum Inhibitory Concentration (MIC) Method A

The MIC test is a test on growth inhibition. Estimation of Minimum Inhibitory Concentration (MIC) is executed in 96 well plates. Through the comparison of bacterial growth curves with positive and negative controls via optical density (OD), different concentrations of given test substances are evaluated.

Bacteria are cultivated under adjusted conditions based on information of the German Collection of Microorganisms and Cell Cultures (DSMZ) and stored in 50% Glycerol prior to use. Afterwards, the following method was applied:

Adding growth medium (according to microorganism) to each well of the microplate.

Adding the test-substances in different concentrations, positive controls (water controls only with medium) and negative controls (benchmarks according to organism).

Adding the microorganism in a concentration of 10⁶ CFU/ml (CFU=colony forming units). Incubation of the microplates at appropriate conditions.

Aerobic microorganism: optical density is continuously measured (microplate reader) during incubation.

Anaerobic microorganism: incubation in an anaerobic chamber, measurement of the optical density at the end of the incubation, comparison of the results with positive and negative controls.

According to resulting growth curves, substance concentrations are labelled either as inhibiting or normal growth and MIC is defined as the lowest concentration where a clear inhibition is visible. Experiments at concentration limits (highest concentration labelled as growth and lowest concentration labelled as inhibiting) are performed at least twice.

B) Minimum Inhibitory Concentration (MIC) Method B

Test conditions: Evaluation of bactericidal and fungicidal activity in qualitative suspension test (VAH-standard methods, 04-2015). 5 ml of the appropriate dilution of the test product is mixed with 0.1 ml of test suspension and mixed well. After the required action times, the mixture was mixed again. Each 0.1 ml were removed and placed in 5 ml of CSL-bouillon without neutralizing agents.

C) Preservative Efficacy Test

Test conditions: Sample (1% test substance in adequate formulation (o/w emulsion)) was inoculated with 0.1 ml of the microbial suspensions, homogenized, and incubated at 20° C.-25° C. Colony count was determined after 2, 7, 14, and 28 days of incubation through plating on adequate solid medium.

2. Results

### Example 1

3-hydroxypropyl caprylate (Error! Reference source not found.), 3-hydroxypropyl undecylenate (Table 2), and glyceryl monoundecylenate (Table 3) were tested on several bacterial and fungal reference strains for application in different body regions using methods A and B (page 16). Obtained results indicate a potential use of both 3-hydroxypropyl caprylate and 3-hydroxypropyl undecylenate in Deodorant-, Skin-, Intimate-, Foot-, and Oral care products. Likewise, several reference strains for preservation showed low MIC values. Likewise, glyceryl monoundecylenate shows low MIC against C. xerosis, indicating at least a compareable application.

TABLE 1
Minimum inhibitory concentrations of 3-hydroxypropyl caprylate
against selected reference bacterial and fungal strains.
Corresponding methods are shown in method section above

| Application | Organism | MIC (ppm) | Method |
|---|---|---|---|
| Deodorant | Corynebacterium xerosis | 500 | A |
| | Staphylococcus epidermidis | 500 | A |
| | Staphylococcus hominis | 1000 | A |
| Preservation | Aspergillus brasiliensis | 1000 | A |
| | Candida albicans | 500 | A |
| | Escherichia coli | 1000 | A |
| | Staphylococcus aureus | 500 | A |
| Skin Care | Peptoniphilus lacrimalis | 1000 | B |
| Womans health | Streptococcus agalactiae | 10000 | B |
| | Gardnerella vaginalis | 500 | B |
| | Fannyhessea vaginae | 500 | B |
| | (Atopobium vaginae) | | |
| Foot Care | Epidermophyton floccosum | 500 | B |
| | Trichophyton rubrum | 500 | B |
| Oral Care | Streptococcus mutans | 5000 | B |
| | Porphyromonas gingivalis | 250 | A |
| | Fusobacterium nucleatum | 250 | A |
| | Prevotella intermedia | 125 | A |

TABLE 2
Minimum inhibitory concentrations (MIC) of 3hydroxypropyl
undecylenate against selected reference bacterial
and fungal strains. Corresponding methods are
shown in method section above

| | | MIC | |
|---|---|---|---|
| Application | Organism | (PPm) | Method |
| Deodorant | Staphylococcus epidermidis | 64 | A |
| | Corynebacterium xerosis | 250 | A |
| Preservation | Aspergillus brasiliensis | 500 | A |
| | Staphylococcus aureus | 500 | A |

TABLE 3
Minimum inhibitory concentrations (MIC) of glyceryl monoundecylenate
against Corynebacterium xerosis. Corresponding methods
are shown in method section above

| | | MIC | |
|---|---|---|---|
| Application | Organism | (PPm) | Method |
| Deodorant | Corynebacterium xerosis | 250 | A |

### Example 2

3-hydroxypropyl caprylate was tested in a potential formulation against the microorganisms Escherichia coli, Pseudomonas aeruginosa, Staphylococcus aureus, and Candida albicans using method C. Resulting colony counts are depicted in Table 4.

TABLE 4
Inhibiting properties of 3-hydroxypropyl caprylate in formulation.
Corresponding methods are shown in method section above

| | Colony counts (cfu/g formulation) | | | | |
|---|---|---|---|---|---|
| Organism | Inoculum | Day 2 | Day 7 | Day 14 | Day 28 |
| E. coli | 4500000 | 15 | >10 | >10 | >10 |
| P. aeruginosa | 4400000 | >10 | >10 | >10 | >10 |
| S. aureus | 3500000 | 250000 | >10 | >10 | >10 |
| C. albicans | 4200000 | 330000 | >10 | >10 | >10 |
| | Relative colony counts (cfu/g formulation) | | | | |
| Organism | Inoculum | Day 2 | Day 7 | Day 14 | Day 28 |
| E. coli | 1.0000 | 0.0000 | 0.0000 | 0.0000 | 0.0000 |
| P. aeruginosa | 1.0000 | 0.0000 | 0.0000 | 0.0000 | 0.0000 |
| S. aureus | 1.0000 | 0.0714 | 0.0000 | 0.0000 | 0.0000 |
| C. albicans | 1.0000 | 0.0786 | 0.0000 | 0.0000 | 0.0000 |

### Example 3

The synergistic activity of 3-hydroxypropyl caprylate and antimicrobial diols against Epidermophyton floccosum was determined by measuring minimum inhibitory concentrations (MIC values) according to DIN 58940 and method B (method section above). The results are shown in Table 5,

Table 6, and Table 7.

In order to calculate the synergy index with MIC values, the Kull equation (I) was used as follows:

SI=(MICₘᵢₓₜᵤᵣₑ×P_{A})/MIC_{A}+(MICₘᵢₓₜᵤᵣₑ×P_{B})/MIC_{B} (I)

wherein

SI is the Synergy Index according to Kull

MIC_{A} is the MIC value for Substance A

MIC_{B} is the MIC value for Substance B

MICₘᵢₓₜᵤᵣₑ is the MIC value for the mixture of substances A and B

P_{A} is the proportion of the substance A in the mixture

P_{B} is the proportion of the substance B in the mixture

TABLE 5
Minimum inhibitory concentrations of 3-hydroxypropyl
caprylate and different antimicrobial diols. Corresponding
methods are shown in method section above

| | | MIC values (ppm) |
|---|---|---|
| Substance Name | CAS number | Epidermophyton floccosum |
| 3-hydroxypropyl caprylate | 102731-54-4 | 500 |
| Pentylene Glycol | 5343-92-0 | 15000 |
| 1,2 Hexanediol | 6920-22-5 | 10000 |
| Caprylyl Glycol | 1117-86-8 | 5000 |

TABLE 6
Minimum inhibitory concentrations of mixtures of 3-hydroxypropyl
caprylate with different antimicrobial diols. Corresponding
methods are shown in method section above

| | | MIC values (ppm): |
|---|---|---|
| Mixtures | Weight ratio | Epidermophyton floccosum |
| 3-hydroxypropyl caprylate | 1:1 | 500 |
| 1,2-Pentanediol | | |
| 3-hydroxypropyl caprylate | 1:1 | 250 |
| 1,2-Hexanediol | | |
| 3-hydroxypropyl caprylate | 1:1 | 250 |
| 1,2-Octanediol | | |

TABLE 7
Synergy Index according to Kull of mixtures of 3-hydroxypropyl
caprylate with different antimicrobial diols

| | | SI |
|---|---|---|
| Mixtures | Weight ratio | Epidermophyton floccosum |
| 3-hydroxypropyl caprylate | 1:1 | 0.517 |
| 1,2-Pentanediol | | |
| 3-hydroxypropyl caprylate | 1:1 | 0.263 |
| 1,2-Hexanediol | | |
| 3-hydroxypropyl caprylate | 1:1 | 0.275 |
| 1,2-Octanediol | | |

The Synergy Index for the combination of 3-hydroxypropyl caprylate with different antimicrobial diols against Epidermophyton floccosum, calculated according to Kull's equation (I) is below 1 for all cases, indicating a synergism between 3-hydroxypropyl caprylate and 1,2-Pentanediol, 1,2-Hexanediol, or 1,2-Octanediol.

To verify that the tested combinations act synergistically against other microorganisms of the natural microbiota of mammalian skin, a corresponding experiment was conducted with Corynebacterium xerosis using the combination of 3-hydroxypropyl caprylate and 1,2-octanediol as an example. The MIC values of 3-hydroxypropyl caprylate and 1,2-octanediol against Corynebacterium xerosis are 500 ppm and 2500 ppm, respectively. For a 1:1 mixture of 3-hydroxypropyl caprylate and 1,2-octanediol a MIC value of 500 ppm was determined, resulting in a Synergy Index of 0.600.

### Example 4

The synergistic activity of 3-hydroxypropyl caprylate and dimethyl phenylbutanol on several bacterial and fungal reference strains for application in either Deodorant application or as preservative was determined by measuring minimum inhibitory concentrations (MIC values) according to DIN 58940 and method A (method section above). The results are shown in Table 8,

Table 9, and Table 10.

TABLE 8
Minimum inhibitory concentrations of 3-hydroxypropyl caprylate and dimethyl
phenylbutanol. Corresponding methods are shown in method section above

| Substance | CAS | MIC values (ppm) | | | |
|---|---|---|---|---|---|
| Name | number | S. epidermidis | A. brasiliensis | E. coli | S. aureus |
| 3-hydroxypropyl | 102731-54-4 | 500 | 1000 | 1000 | 500 |
| caprylate | | | | | |
| Dimethyl | 2035-93-0 | 1000 | 2500 | 2500 | 1000 |
| Phenylbutanol | | | | | |

TABLE 9
Minimum inhibitory concentrations of mixtures of 3-hydroxypropyl
caprylate and dimethyl phenylbutanol. Corresponding
methods are shown in method section above.

| | Weight | MIC values (ppm) | | | |
|---|---|---|---|---|---|
| Mixtures | Ratio | S. epidermidis | A. brasiliensis | E. coli | S. aureus |
| 3-hydroxypropyl | 1:1 | 500 | 1000 | 1000 | 500 |
| caprylate | | | | | |
| dimethyl | | | | | |
| phenylbutanol | | | | | |

TABLE 10
Synergy Index according to Kull of mixtures of 3-
hydroxypropyl caprylate and dimethyl phenylbutanol

| | Weight | SI | | | |
|---|---|---|---|---|---|
| Mixtures | Ratio | S. epidermidis | A. brasiliensis | E. coli | S. aureus |
| 3-hydroxypropyl | 1:1 | 0.75 | 0.70 | 0.70 | 0.75 |
| caprylate dimethyl | | | | | |
| phenylbutanol | | | | | |

The Synergy Index SI for the combination of 3-hydroxypropyl caprylate and dimethyl phenylbutanol against the strain related to deodorant applications (Staphylococcus epidermidis) and strains related to preservative function (Aspergillus brasiliensis, Escherichia coli, Staphylococcus aureus) calculated according to Kull's equation (I) is below 1 in all cases tested, indicating a synergism between both substances.

### Example 5

The synergistic activity of 3-hydroxypropyl caprylate and glyceryl caprylate on several bacterial and fungal reference strains for application in either Deodorant application or as preservative was determined by measuring minimum inhibitory concentrations (MIC values) according to DIN 58940 and method B (method section above).

The results are shown in Table 11, Table 12, and Table 13.

TABLE 11
Minimum inhibitory concentrations of 3-hydroxypropyl
caprylate and Glyceryl Caprylate

| Substance | CAS | MIC values (ppm) | | | |
|---|---|---|---|---|---|
| name | number | S. hominis | C. xerosis | A. brasiliensis | E. coli |
| 3-hydroxypropyl | 102731-54-4 | 1000 | 500 | 1000 | 1000 |
| caprylate | | | | | |
| glyceryl caprylate | 26402-26-6 | 1000 | 1000 | 2500 | 2500 |

TABLE 12
Minimum inhibitory concentrations of mixtures of 3-hydroxypropyl caprylate and
Glyceryl Caprylate. Corresponding methods are shown in method section above

| | Weight | MIC values (ppm) | | | |
|---|---|---|---|---|---|
| Mixtures | Ratio | S. hominis | C. xerosis | A. brasiliensis | E. coli |
| 3-hydroxypropyl caprylate | 1:1 | 500 | 500 | 1000 | 1000 |
| glyceryl caprylate | | | | | |

TABLE 13
Synergy Index according to Kull of mixtures of 3-
hydroxypropyl caprylate and glyceryl caprylate

| | Weight | SI | | | |
|---|---|---|---|---|---|
| Mixtures | Ratio | S. hominis | C. xerosis | A. brasiliensis | E. coli |
| 3-hydroxypropyl caprylate | 1:1 | 0.5 | 0.75 | 0.7 | 0.7 |
| glyceryl caprylate | | | | | |

The Synergy Index SI for the combination of 3-hydroxypropyl caprylate and glyceryl caprylate against the strain related to deodorant applications (Staphylococcus hominis) and Aspergillus brasiliensis as a strain related to preservative function, calculated according to Kull's equation (I), is below 1 in all cases tested, indicating a synergism between both substances.

### Example 6

The synergistic activity of 3-hydroxypropyl caprylate and Farnesol on Corynebacterium xerosis for application in deodorant was determined by measuring minimum inhibitory concentrations (MIC values) according to DIN 58940 and method B (method section above).

The results are shown in Table 14, Table 15, and Table 16.

TABLE 14
Minimum inhibitory concentrations of 3-hydroxypropyl
caprylate and Farnesol. Corresponding methods
are shown in method section above

| Substance | | MIC values (ppm) |
|---|---|---|
| name | CAS number | C. xerosis |
| 3-hydroxypropyl caprylate | 102731-54-4 | 5000 |
| Farnesol | 4602-84-0 | 16 |

TABLE 15
Minimum inhibitory concentrations of mixtures of
3-hydroxypropyl caprylate and Farnesol. Corresponding
methods are shown in method section above

| | | MIC values (ppm) |
|---|---|---|
| Mixtures | Weight Ratio | C. xerosis |
| 3-hydroxypropyl caprylate | 1:1 | 16 |
| Farnesol | | |

TABLE 16
Synergy Index of mixtures of 3-hydroxypropyl
caprylate and Farnesol

| | | SI |
|---|---|---|
| Mixtures | Weight Ratio | C. xerosis |
| 3-hydroxypropyl caprylate | 1:1 | 0.516 |
| Farnesol | | |

The Synergy Index SI for the combination of 3-hydroxypropyl caprylate and Farnesol against the test strain related to deodorant applications, calculated according to Kull's equation (I) is 0.516 for Corynebacterium xerosis, indicating synergism between both substances.

### Example 7

Additional tests were performed with human sebaceous glands in order to verify the modulatory activity of a compound on sebum secretion. For this sebaceous glands had been micro-dissected and cultured with medium. The medium without any supplement served as untreated control. Medium with 5 μM Capsaicin served as positive control. Additionally, culture medium was supplemented with different concentrations of 3-hydroxypropyl caprylate. After six days the sebaceous glands were collected and lipids and proteins quantified. The total lipid amount being representative of sebum production was obtained by normalizing the quantified lipids upon the quantified proteins (i.e. mg of lipids/mg of proteins).

As shown in FIGS. 1 and 2, a concentration dependent reduction of total lipids was found. This demonstrates the sebum reduction activity of 3-hydroxypropyl caprylate. As sebum contains compounds that serve as substrate for microbial growth, sebum reduction can also reduce the amount of microbiota on the skin. Based on these results, it is assumed that the fatty acid esters disclosed herein have microbiota balancing or reducing properties indirectly via sebum reduction.

### Example 8: Formulation Examples

TABLE 17
Composition of perfume oil 1 (PO1, amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| Benzylacetat | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2.4 10% IPM | 2 |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 18
Composition of perfume oil 2 (PO2, amounts in ‰‰ b.w.)

| Ingredients | Amount |
|---|---|
| Acetophenone 10% in DPG | 10 |
| n-Undecanal | 5 |
| Aldehyde C14, so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl-phenyl)propanal) | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 19
Composition of perfume oil 3 (PO3, amounts in ‰ b.w.)

| Ingredients | Amount |
|---|---|
| Benzyl acetate | 60 |
| Citronellyl acetate | 60 |
| Cyclamenaldehyde (2-methyl-3-(4-isopropylphenyl)propanal | 20 |
| Dipropylene glycol (DPG) | 60 |
| Ethyllinalool | 40 |
| Florol (2-isobutyl-4-methyltetrahydro-2H-pyran-4-ol) | 30 |
| Globanone ® [(E/Z)-8-cyclohexadecen-1-one] | 180 |
| Hedione ® (methyldihydrojasmonate) | 140 |
| Hexenyl salicylate, cis-3 | 10 |
| Vertocitral (2,4-dimethyl-3-cyclohexenecarboxaldehyde) | 5 |
| Hydratropaldehyde, 10% in DPG | 5 |
| Isodamascone (1-(2,4,4-trimethyl-2-cyclohexen-1-yl)-2-buten-1- | 5 |
| one, 10% in DPG | |
| Isomuscone (cyclohexadecanone) | 40 |
| Jacinthaflor (2-methyl-4-phenyl-1,3-dioxolane) | 10 |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehyde | 5 |
| 2-Phenylethyl alcohol | 82 |
| Tetrahydrogeraniol | 13 |
| 2,2-Dimethyl-3-cyclohexyl-1-propanol | 80 |
| Total: | 1000 |

TABLE 20
Composition of perfume oil 4 (PO4, amounts in ‰ b.w.)

| Ingredients | Amount |
|---|---|
| AMBRETTOLIDE (MACRO) | 10 |
| AMBROXIDE, 10% in IPM | 10 |
| BENZYL ACETATE | 20 |
| BENZYL SALICYLATE | 15 |
| BERGAMOT OIL, bergapten-free | 60 |
| CALONE ® 1951, 10% in DPG | 15 |
| COUMARIN | 5 |
| CYCLOGALBANATE ®^{,} 10% in DPG | 10 |
| ALPHA-DAMASCONE, 1% in DPG | 20 |
| DIHYDROMYRCENOL | 10 |
| ETHYL LINALOOL | 75 |
| ETHYL LINALYLACETATE | 50 |
| ETHYL MALTOL, 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 21
Composition of perfume oil 5 (PO5, amounts in ‰ b.w.)

| Ingredients | Amount |
|---|---|
| AMAROCITE ® | 10 |
| AMBROCENIDE ®, 10% in DPG | 5 |
| AMBROXIDE | 15 |
| AURELIONE ® (7/8-Cyclohexadecenone) (MACRO) | 70 |
| BERGAMOT OIL, bergapten-free | 90 |
| CALONE ® 1951, 10% in DPG | 20 |
| CARAWAY OIL | 10 |
| CITRAL | 20 |
| COUMARIN | 10 |
| ALPHA-DAMASCONE, 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE(MACRO) | 5 |
| SANDRANOL ® | 10 |
| VANILLIN 10% in DPG | 5 |
| DIPROPYLENE GLYCOL | 30 |
| TOTAL | 1000 |

The perfume oils PO1, PO2, PO3, PO4, or PO5 from the above examples were worked separately in each case into the formulations presented below.

Cosmetic formulations (compositions)—amounts are indicated as % by weight for all formulations.

TABLE 22
Cream o/w

| Ingredients | INCI | Amount |
|---|---|---|
| Dracorin ® CE | Glyceryl Stearate Citrate | 1.0 |
| Lanette ® O | Cetearyl Alcohol | 2.0 |
| Cutina ® GMS-V | Glyceryl Stearate | 1.0 |
| Tegosoft ® MM | Myristyl Myristate | 1.0 |
| Xiameter ® PMX-0246, | Cyclohexasiloxane (and) | 0.5 |
| Cyclosiloxane | Cyclopentasiloxane | |
| Dragoxat ® 89 | Ethylhexyl Isononanoate | 2.0 |
| PCL-Liquid 100 | Cetearyl Ethylhexanoate | 4.0 |
| Neutral Oil | Caprylic/Capric Triglyceride | 4.0 |
| Carbopol ® Ultrez 21 | Acrylates/C10-30 Alkyl Acrylate | 0.2 |
| | Crosspolymer | |
| Keltrol ® CG-T | Xanthan Gum | 0.1 |
| Water | Water (Aqua) | ad 100 |
| Glycerol 99.5 P. | Glycerol | 3.0 |
| Hydrolite CG | Caprylyl Glycol | 0.2 |
| 1,2-Propylene | Propylene Glycol | 2.0 |
| Glycol 99 P GC | | |
| Sodium Benzoate | Sodium Benzoate | 0.1 |
| Sodium Hydroxide 10% | Sodium Hydroxide | 0.5 |
| solution | | |
| Perfume oil PO1, PO2, | Perfume | 0.3 |
| PO3, PO4, or PO5 | | |
| Euxyl ® K702 | Dehydroacetic Acid, Benzoic Acid, | 0.3 |
| | Phenoxyethanol, Polyaminopropyl | |
| | Biguanide, Ethylhexylglycerin | |
| 3-Hydroxypropyl | Propanediol Caprylate | 0.3 |
| caprylate | | |
| 3-Hydroxypropyl | Hydroxypropyl undecylenate | 0.1 |
| undecylenate | | |

TABLE 23
Hand and body cream

| Ingredients | INCI | Amount |
|---|---|---|
| Dracorin ® GOC | Glyceryl Oleate Citrate, | 2.0 |
| | Caprylic/Capric Triglyceride | |
| PCL-Solid | Stearyl Heptanoate, Stearyl | 2.5 |
| | Caprylate | |
| Lanette ® O | Cetearyl Alcohol | 1.5 |
| Cutina ® GMS-V | Glyceryl Stearate | 1.0 |
| Dragoxat ® 89 | Ethylhexyl Isononanoate | 3.0 |
| PCL-Liquid 100 | Cetearyl Ethylhexanoate | 7.0 |
| Isodragol ® | Triisononanoin | 4.0 |
| Xiameter ® PMX-0345 | Cyclopentasiloxane (and) | 0.5 |
| Cyclosiloxane | Cyclohexasiloxane | |
| Water | Water (Aqua) | ad 100 |
| Carbopol ® Ultrez 21 | Acrylates/C10-30 Alkyl Acrylate | 0.2 |
| | Crosspolymer | |
| Keltrol ® CG-RD | Xanthan Gum | 0.1 |
| Glycerol 85 P. | Glycerol | 3.0 |
| DragoBetaGlucan | Water (Aqua). Butylene Glycol, | 1.5 |
| | Glycerol, Avena Sativa (Oat) | |
| | Kernel Extract | |
| Potassium Sorbate | Potassium Sorbate | 0.1 |
| Hydrolite-6 | 1,2 Hexanediol | 1.0 |
| Sodium Hydroxide 10% | Sodium Hydroxide | 0.5 |
| solution | | |
| Perfume oil PO1, PO2, | Fragrance | 0.2 |
| PO3, PO4, or PO5 | | |
| 3-Hydroxypropyl caprylate | Propanediol Caprylate | 0.2 |
| Glyceryl monocaprylate | Glyceryl caprylate | 0.05 |

TABLE 24
Daily face cream SPF 20

| Ingredients | Amount |
|---|---|
| SymOcide PH | 1 |
| Phenoxyethanol, Hydroxyacetophenone, Caprylyl Glycol, Water | |
| (Aqua) | |
| Ascorbyl Palmitate | 0.1 |
| Ascorbyl Palmitate | |
| Biotive L-Arginine | 0.2 |
| Arginine | |
| Buriti oil | 1 |
| Mauritia Flexuosa Fruit Oil | |
| Cocoa butter | 2 |
| Theobroma Cacao (Cocoa) Seed Butter | |
| Dimethicone | 0.5 |
| Dimethicone | |
| Disodium EDTA | 0.1 |
| Disodium EDTA | |
| Dragosantol 100 | 0.1 |
| Bisabolol | |
| Dragoxat 89 | 5 |
| Ethylhexyl Isononanoate | |
| Emulsiphos | 2 |
| Potassium Cetyl Phosphate, Hydrogenated Palm Glycerides | |
| Extrapone Corail | 1 |
| Glycerin, Aqua, Hydrolyzed Corallina Officinalis | |
| Glycerin | 3 |
| Glycerin | |
| Isoadipate | 5 |
| Diisopropyl Adipate | |
| Jojoba Wax Flakes | 1 |
| Hydrogenated Jojoba Oil | |
| Keltrol CG-T | 0.1 |
| Xanthan Gum | |
| Lanette O | 5 |
| Cetearyl Alcohol | |
| Lanette 16 | 1 |
| Cetyl Alcohol | |
| Lanette 22 | 1 |
| Behenyl Alcohol | |
| Neo Heliopan 357 | 3 |
| Butyl Methoxydibenzoylmethane | |
| Neo Heliopan HMS | 10 |
| Homosalate | |
| Neo Heliopan Hydro used as a 25% aqueous solution neutralized | 8 |
| by arginine | |
| Phenylbenzimidazole Sulfonic Acid | |
| Neo Heliopan OS | 5 |
| Ethylhexyl Salicylate | |
| Orgasol Caresse | 1 |
| Polyamide-5 | |
| Perfume oil PO1, PO2, PO3, PO4, or PO5 | 0.1 |
| Shea butter | 3 |
| Butyrospermum Parkii (Shea) Butter | |
| Simugel EG | 1 |
| Sodium Acrylate/Sodium Acryloyldimethyl Taurate Copolymer, | |
| Isohexadecane, Polysorbate 80 | |
| SymFinity 1298 | 0.1 |
| Echinacea Purpurea Extract | |
| SymDiol 68 | 0.5 |
| 1,2 Hexanediol, Caprylyl Glycol | |
| SymMatrix | 0.1 |
| Maltodextrin, Rubus Fructicosus (Blackberry) Leaf Extract | |
| SymSitive 1609 | 1 |
| Pentylene Glycol, 4-t-Butylcyclohexanol | |
| Tegosoft TN | 4 |
| C12-15 Alkyl Benzoate | |
| 3-Hydroxypropyl caprylate | 0.3 |
| Glyceryl monoundecylenate | 0.1 |
| Water | ad 100 |
| Aqua | |

TABLE 25
w/o night cream

| Ingredients | INCI | Amount |
|---|---|---|
| 3-Hydroxypropyl | Propanediol Caprylate | 0.15 |
| caprylate | | |
| Glyceryl | Glyceryl caprylate | 0.15 |
| monocaprylate | | |
| 3-Hydroxypropyl | Hydroxypropyl undecylenate | 0.1 |
| undecylenate | | |
| Aloe Vera Gel | Water (Aqua), Aloe Barbadensis Leaf | 3.0 |
| Concentrate 10/1 * | Juice | |
| Alugel 34 TH | Aluminium Stearate | 1.0 |
| Dragosan W/O P* | Sorbitan Isostearate, Hydrogenated | 6.0 |
| | Castor Oil, Ceresin, Beeswax (Cera | |
| | Alba) | |
| Dragosantol ® 100* | Bisabolol | 0.2 |
| Extrapone ® | Propylene Glycol, Hamamelis | 1.0 |
| Witch Hazel | Virginiana (Witch Hazel) Water, | |
| Distillate | Water (Aqua), Hamamelis Virginiana | |
| colourless | (Witch Hazel) Extract | |
| Perfume oil PO1, PO2, | Fragrance | 0.4 |
| PO3, PO4, or PO5 | | |
| Glycerol 85% | Glycerin | 2.0 |
| Hydrolite-5 | Pentylene Glycol | 0.5 |
| Karion F | Sorbitol | 2.0 |
| Magnesium Chloride | Magnesium Chloride | 0.7 |
| PCL Liquid 100 | Cetearyl Ethylhexoate | 12.0 |
| Retinyl Palmitate in Oil | Retinyl Palmitate | 0.2 |
| Sun Flower Oil | Helianthus Annuus (Sunflower) Seed | 5.0 |
| | Oil | |
| Sweet Almond Oil | Prunus dulcis | 5.0 |
| SymMatrix ® | Maltodextrin, Rubus Fruticosus | 1.0 |
| | (Blackberry) Leaf Extract | |
| SymOcide PS | Phenoxyethanol, Decylene glycol, | 1.0 |
| | 1,2-Hexanediol | |
| SymVital ® AgeRepair | Zingiber Officinale (Ginger) Root | 0.1 |
| | Extract | |
| Tocopherol Acetate | Tocopheryl Acetate | 3.0 |
| Water (demineralized) | Water (Aqua) | ad 100 |

TABLE 26
Body lotion

| Ingredients | Amount |
|---|---|
| Cetearyl Alcohol | 2.0 |
| Ethylhexyl Isononanoate | 5.0 |
| Cetearyl Ethylhexanoate, Isopropyl Myristate | 3.0 |
| Glyceryl Oleate Citrate, Caprylic/Capric Triglyceride | 4.0 |
| Water (Aqua) | ad 100 |
| Pentylene Glycol | 3.0 |
| Carbomer | 0.3 |
| Sodium Benzoate | 0.1 |
| Propylene Glycol | 5.0 |
| Sodium Hydroxide 30% solution | 0.3 |
| Perfume oil PO1, PO2, PO3, PO4, or PO5 | 0.3 |
| Triethylene Glycol. Imidazolidinyl Urea, Methylparaben, | 0.3 |
| Propylparaben, Dehydroacetic Acid | |
| 3-Hydroxypropyl caprylate | 0.2 |
| 3-Hydroxypropyl undecylenate | 0.2 |

TABLE 27
Antibacterial body lotion, sprayable

| Ingredients | INCI | Amount |
|---|---|---|
| 3-Hydroxypropyl | Propanediol Caprylate | 0.15 |
| caprylate | | |
| Glyceryl | Glyceryl caprylate | 0.15 |
| monocaprylate | | |
| 3-Hydroxypropyl | Hydroxypropyl undecylenate | 0.05 |
| undecylenate | | |
| Glyceryl | Glyceryl undecylenate | 0.05 |
| monoundecylenate | | |
| Triethyl Citrate | Triethyl Citrate | 0.2 |
| 2.4-Hexadienoic acid, | Sorbic acid, potassium salt | 0.2 |
| potassium salt | | |
| Dow Corning 345 Fluid | Cyclomethicone | 0.5 |
| Dracorin ® GOC | Glyceryl Oleate Citrate, | 2.0 |
| | Caprylic/Capric Triglyceride | |
| Drago-Calm | Water, Glycerin, Avena Sativa | 1.0 |
| | (Oat) Kernel Extract | |
| Dragosantol ® 100* | Bisabolol | 0.1 |
| Perfume oil PO1, PO2, | Fragrance | 0.3 |
| PO3, PO4, or PO5 | | |
| Hydrolite ®-5 | Pentylene Glycol | 5.0 |
| Neutral Oil | Caprylic/Capric Triglyceride | 4.0 |
| Paraffin Oil | Mineral Oil | 4.0 |
| PCL Liquid 100 | Cetearyl Ethylhexoate | 7.0 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% sol.) | | |
| SymDeo ® MPP | Dimethyl Phenylbutanol | 0.5 |
| SymRelief ® 100 | Bisabolol, Zingiber Officinale | 0.1 |
| | (Ginger) Root Extract | |
| Water (demineralized) | Water (Aqua) | ad 100 |

TABLE 28
Aseptic wound cream

| Ingredients | Amount |
|---|---|
| Sorbitan Isostearate, Hydrogenated Castor Oil, | 6.0 |
| Ceresin, Beeswax (Cera Alba) | |
| Petrolatum | 21.0 |
| Cera Alba | 5.0 |
| Cetearyl Alcohol | 7.0 |
| Prunus Dulcis | 7.0 |
| Lanolin | 5.0 |
| Paraffinum Liquidum | 12.0 |
| Perfume oil PO1, PO2, PO3, PO4, or PO5 | 0.3 |
| Water (Aqua) | ad 100 |
| Panthenol | 7.0 |
| Magnesium Sulfate | 0.7 |
| Pentylene Glycol | 1.0 |
| Tocopheryl Acetate | 1.0 |
| Octenidine dihydrochloride | 0.1 |
| Phenoxyethanol | 0.5 |
| 3-Hydroxypropyl caprylate | 0.4 |
| Glyceryl monocaprylate | 0.2 |

TABLE 29
Anti acne balm

| Ingredients | INCI | Amount |
|---|---|---|
| 3-Hydroxypropyl | Propanediol Caprylate | 0.3 |
| caprylate | | |
| 3-Hydroxypropyl | Hydroxypropyl undecylenate | 0.2 |
| undecylenate | | |
| Abil 350 | Dimethicone | 1.0 |
| Allantoin | Allantoin | 0.1 |
| Aloe Vera Gel | Water (Aqua), Aloe Barbadensis | 3.0 |
| Concentrate 10/1 * | Leaf Juice | |
| Azelaic Acid | Azelaic Acid | 5.0 |
| Cetiol OE | Dicaprylyl Ether | 4.0 |
| Cetiol SB 45 | Butyrospermum Parkii | 1.0 |
| | (Shea Butter) | |
| D-Panthenol | Panthenol | 1.0 |
| SymClariol | Decylene Glycol | 0.1 |
| Emulsiphos ® | Potassium Cetyl Phosphate, | 2.0 |
| | Hydrogenated Palm Glycerides | |
| Perfume oil PO1, PO2, | Fragrance | 0.2 |
| PO3, PO4, or PO5 | | |
| Frescolat ®ML cryst. | Menthyl Lactate | 0.8 |
| Glycerol 85% | Glycerin | 4.0 |
| Hydroviton ® PLUS | Water, Pentylene Glycol, Glycerin, | 1.0 |
| | Fructose, Urea, Citric Acid, Sodium | |
| | Hydroxide, Maltose, Sodium PCA, | |
| | Sodium Chloride, Sodium Lactate, | |
| | Trehalose, Allantoin, Sodium | |
| | hyaluronate, Glucose | |
| Lara Care A-200 | Galactoarabinan | 0.3 |
| Pemulen TR-2 | Acrylates/C10-30 Alkyl Acrylate | 0.2 |
| | Crosspolymer | |
| Sodium Hydroxide (10% | Sodium Hydroxide | 0.4 |
| sol.) | | |
| SymOcide PH | Hydroxyacetophenone, | 1.0 |
| | Phenoxyethanol, Caprylyl glycol, | |
| | Aqua | |
| Tegosoft TN | C12-15 Alkyl Benzoate | 5.0 |
| Tocopherol Acetate | Tocopheryl Acetate | 0.5 |
| Water (demineralized) | Water (Aqua) | ad 100 |

TABLE 30
Barrier repair cream

| Ingredients | INCI | Amount |
|---|---|---|
| 3-Hydroxypropyl | Propanediol Caprylate | 0.1 |
| caprylate | | |
| Glyceryl | Glyceryl caprylate | 0.1 |
| monocaprylate | | |
| 3-Hydroxypropyl | Hydroxypropyl undecylenate | 0.1 |
| undecylenate | | |
| Glyceryl | Glyceryl undecylenate | 0.1 |
| monoundecylenate | | |
| Abil 350 | Dimethicone | 0.5 |
| Allantoin | Allantoin | 0.25 |
| Ceramide BIO* | Cetylhydroxyproline Palmitamide | 0.5 |
| Dracorin ® CE | Glyceryl Stearate Citrate | 1.5 |
| Dragoxat ® 89 | Ethylhexyl Isononanoate | 2.0 |
| Emulsiphos ® | Potassium Cetyl Phosphate, | 2.0 |
| | Hydrogenated Palm Glycerides | |
| Extrapone ® | Glycerin, Water (Aqua), | 0.5 |
| Rosemary GW | Rosmarinus officinalis | |
| | (Rosemary) Leaf Extract | |
| Perfume oil PO1, PO2, | Fragrance | 0.1 |
| PO3, PO4, or PO5 | | |
| Glycerol 85% | Glycerin | 3.0 |
| Glyceryl Stearate | Glyceryl Stearate | 2.0 |
| Hydroviton ® 24 | Water, Glycerin, Sodium Lactate, TEA | 1.0 |
| | Lactate, Serine, Lactic Acid, Urea, | |
| | Sorbitol, Sodium Chloride, Lauryl | |
| | Diethylenedi-aminoglycine, Lauryl | |
| | Aminopropyl-glycine, Allantoin | |
| Hydrolite- 5 Green | Pentylene Glycol | |
| Isodragol ® | Triisononanoin | 3.0 |
| Lanette O | Cetearyl Alcohol | 2.0 |
| NaOH 10% sol. | Sodium Hydroxide | 0.3 |
| Neutral Oil | Caprylic/Capric Triglyceride | 10.0 |
| SymCalmin ® | Pentylene Glycol, Butylene Glycol, | 1.0 |
| | Hydroxyphenyl Propamidobenzoic | |
| | Acid | |
| SymRepair ® 100 | Hexyldecanol, Bisabolol, | 2.0 |
| | Cetylhydroxyproline Palmitamide, | |
| | Stearic Acid, Brassica Campestris | |
| | (Rapeseed) Sterols | |
| SymTriol | Caprylyl glycol, 1,2-Hexanediol, | 1.0 |
| | Methylbenzyl alcohol | |
| Tegosoft PC 31 | Polyglyceryl 3- Caprate | 0.3 |
| Tocopherol Acetate | Tocopheryl Acetate | 0.3 |
| Water (demineralized) | Water (Aqua) | ad 100 |

TABLE 31
Skin soothing lotion

| Ingredients | INCI | Amount |
|---|---|---|
| 3-Hydroxypropyl | Propanediol Caprylate | 0.3 |
| caprylate | | |
| Glyceryl | Glyceryl caprylate | 0.05 |
| monocaprylate | | |
| 3-Hydroxypropyl | Hydroxypropyl undecylenate | 0.2 |
| undecylenate | | |
| Glyceryl | Glyceryl undecylenate | 0.05 |
| monoundecylenate | | |
| Abil 350 | Dimethicone | 2.0 |
| Allantoin | Allantoin | 0.2 |
| Carbopol Ultrez-10 | Carbomer | 0.1 |
| Ceramide BIO* | Cetylhydroxyproline Palmitamide | 0.1 |
| Citric Acid 10% sol. | Citric Acid | 0.4 |
| Emulsiphos ® | Potassium Cetyl Phosphate, | 2.0 |
| | Hydrogenated Palm Glycerides | |
| Extrapone ® | Glycerin, Water (Aqua), Camellia | 0.2 |
| Green Tea GW | Sinensis Leaf Extract | |
| Extrapone ® | Glycerin, Water (Aqua), Rosmarinus | 0.3 |
| Rosemary GW | officinalis (Rosemary) Leaf Extract | |
| Perfume oil PO1, PO2, | Fragrance | 0.3 |
| PO3, PO4, or PO5 | | |
| Glycerol 85% | Glycerin | 2.0 |
| Glyceryl Stearate | Glyceryl Stearate | 2.0 |
| Isodragol ® | Triisononanoin | 2.0 |
| Keltrol RD | Xanthan Gum | 0.1 |
| Lanette O | Cetearyl Alcohol | 3.0 |
| Neo PCL wssl. N | Trideceth-9. PEG-5 Ethylhexanoate, | 1.0 |
| | Water | |
| PCL Liquid 100 | Cetearyl Ethylhexanoate | 5.0 |
| PCL Solid | Stearyl Heptanoate, Stearyl Caprylate | 2.0 |
| Propylene Glycol | Propylene Glycol | 5.0 |
| Sodium Hydroxide | Sodium Hydroxide | 0.3 |
| (10% sol.) | | |
| SymCalmin ® | Pentylene Glycol, Butylene Glycol, | 2.0 |
| | Hydroxyphenyl Propamidobenzoic | |
| | Acid | |
| SymMatrix ® | Maltodextrin, Rubus Fruticosus | 0.1 |
| | (Blackberry) Leaf Extract | |
| SymSave H | Hydroxyacetophenone | 0.4 |
| 2-Phenoxyethyl Alcohol | Phenoxyethanol | 0.4 |
| SymSitive ®1609 | Pentylene Glycol, 4-t- | 1.5 |
| | Butylcyclohexanol | |
| Water (demineralized) | Water (Aqua) | ad 100 |

TABLE 32
Baby Nappy Rash Cream w/o

| Ingredients | Amount |
|---|---|
| SymOcide PH | 1 |
| Phenoxyethanol, Hydroxyacetophenone, Caprylyl Glycol, Water | |
| (Aqua) | |
| Cupuaçu butter | 1 |
| Theobroma Grandiflorum Seed Butter | |
| Cutina HR Powder | 1.5 |
| Hydrogenated Castor Oil | |
| Dehymuls PGPH | 5 |
| Polyglyceryl-2 Dipolyhydroxystearate | |
| Glycerin | 5 |
| Glycerin | |
| Jojoba oil | 5 |
| Simmondsia Chinensis (Jojoba) Seed Oil | |
| Magnesium Sulfate Hepta Hydrate | 0.5 |
| Magnesium Sulfate | |
| Monomuls 90-O18 | 1 |
| Glyceryl Oleate | |
| Neutral oil | 8 |
| Caprylic/capric triglyceride | |
| PCL Liquid 100 | 5 |
| Cetearyl Ethylhexanoate | |
| SymCalmin | 1 |
| Butylene Glycol, Pentylene Glycol, Hydroxyphenyl | |
| Propamidobenzoic Acid | |
| Tamanu oil | 0.2 |
| Calophyllum Inophyllum Seed Oil | |
| Tetrasodium EDTA | 0.1 |
| Tetrasodium EDTA | |
| Titan dioxide | 4 |
| Titan dioxide | |
| Water | ad 100 |
| Aqua | |
| Wheat germ oil | 2 |
| Triticum Vulgare (Wheat) Germ Oil | |
| Zinc oxide | 10 |
| Zinc oxide | |
| 3-Hydroxypropyl caprylate | 0.3 |
| 3-Hydroxypropyl undecylenate | 0.15 |

TABLE 33
Skin lightening day cream o/w

| Ingredients | INCI | Amount |
|---|---|---|
| 3-Hydroxypropyl | Propanediol Caprylate | 0.1 |
| caprylate | | |
| Glyceryl | Glyceryl caprylate | 0.1 |
| monocaprylate | | |
| 3-Hydroxypropyl | Hydroxypropyl undecylenate | 0.05 |
| undecylenate | | |
| Glyceryl | Glyceryl undecylenate | 0.05 |
| monoundecylenate | | |
| Abil 350 | Dimethicone | 0.5 |
| Dracorin ® CE | Glyceryl Stearate Citrate | 2.5 |
| Dracorin ® GOC | Glyceryl Oleate Citrate, | 0.5 |
| | Caprylic/Capric Triglyceride | |
| Drago-Beta-Glucan | Water (Aqua), Butylene | 0.3 |
| | Glycol, Glycerin, | |
| | Avena Sativa (Oat) | |
| | Kernel Extract | |
| Dragosantol ® 100* | Bisabolol | 0.2 |
| Perfume oil PO1, PO2, | Fragrance | 0.1 |
| PO3, PO4, or PO5 | | |
| Frescolat ®MGA | Menthone Glycerol Acetal | 0.5 |
| Glycerol 85% | Glycerin | 3.0 |
| Isopropyl Palmitate | Isopropyl Palmitate | 4.0 |
| Keltrol RD | Xanthan Gum | 0.2 |
| Lanette 16 | Cetyl Alcohol | 1.0 |
| Neo Heliopan ® AV | Ethyl hexyl Methoxy-cinnamate | 5.0 |
| Neutral Oil | Caprylic/Capric Triglyceride | 6.0 |
| PCL Liquid 100 | Cetearyl Ethylhexoate | 3.0 |
| Sodium Benzoate | Sodium Benzoate | 0.1 |
| Symdiol ®68T | 1,2-Hexanediol, Caprylylglycol, | 0.5 |
| | Tropolone | |
| SymVital ® AgeRepair | Zingiber Officinale | 0.1 |
| | (Ginger) Root Extract | |
| SymWhite ®377 | Phenylethyl Resorcinol | 0.5 |
| Water (demineralized) | Water (Aqua) | ad 100 |

TABLE 34
Shampoo

| Ingredients | Amount |
|---|---|
| 4-Hydroxyacetophenone (SymSave H) | 0.3 |
| Hydroxyacetophenone | |
| Antil 127 | 0.5 |
| PEG-120 Methyl Glucose Dioleate | |
| Brazilian nut oil | 0.5 |
| Bertholletia Excelsa Seed Oil | |
| Cocamidopropyl Betaine 38% | 5 |
| Cocamidopropyl Betaine | |
| Octopirox | 0.3 |
| Piroctone olamine | |
| Dragoderm | 0.5 |
| Glycerin, Triticum Vulgare Gluten, Aqua | |
| Fragrance | 0.5 |
| Perfum | |
| Glycerin | 0.5 |
| Glycerin | |
| Jojoba oil | 0.5 |
| Simmondsia Chinensis (Jojoba) Seed Oil | |
| Marlinat 242/90 M | 15 |
| MIPA Laureth Sulfate, Propylene Glycol | |
| Marlowet CG | 2 |
| PEG-18 Castor Oil Dioleate | |
| Plantacare 1200 UP | 0.5 |
| Lauryl Glucoside | |
| Polyquaternium-10 | 0.3 |
| Polyquaternium-10 | |
| Sodium Chloride | 1.5 |
| Sodium Chloride | |
| SymCalmin | 1 |
| Butylene Glycol, Pentylene Glycol, Hydroxyphenyl | |
| Propamidobenzoic Acid | |
| SymOcide PS | 0.8 |
| Phenoxyethanol, Decylene Glycol, 1,2-Hexanediol | |
| 3-Hydroxypropyl caprylate | 0.4 |
| 3-Hydroxypropyl undecylenate | 0.2 |
| Water | ad 100 |
| Aqua | |

TABLE 35
Anti dandruff shampoo

| Ingredients | INCI | Amount |
|---|---|---|
| 3-Hydroxypropyl | Propanediol Caprylate | 0.3 |
| caprylate | | |
| 3-Hydroxypropyl | Hydroxypropyl undecylenate | 0.15 |
| undecylenate | | |
| Aloe Vera Gel | Water (Aqua), Aloe Barbadensis | 0.5 |
| Concentrate 10/1 * | Leaf Juice | |
| Abrasive/Exfoliant | Perlite | 0.3 |
| Cellulose fibre | Microcrystalline Cellulose | 0.1 |
| Avocado oil | Persea Gratissima | 0.5 |
| | (Avocado) Oil | |
| Citric Acid 10% sol. | Citric Acid | 0.3 |
| Comperlan 100 | Cocamide ME A | 0.5 |
| Crinipan AD | Climbazole | 0.2 |
| Dragoderm ® | Glycerin, Triticum Vulgare | 2.0 |
| | (Wheat) Gluten, Water (Aqua) | |
| Perfume oil PO1, PO2, | Fragrance | 0.5 |
| PO3, PO4, or PO5 | | |
| Genapol LRO liquid | Sodium Laureth Sulfate | 37.0 |
| Merquat 550 | Polyquaternium-7 | 0.5 |
| Xylityl Caprylate | Xylityl Caprylate | 0.5 |
| Sodium Chloride | Sodium Chloride | 1.0 |
| Hydrolite-5 Green | Pentylene Glycol | 0.5 |
| SymSave ® H | Hydroxyacetophenone | 0.8 |
| Tego Betain L7 | Cocamidopropyl Betaine | 6.0 |
| Water (demineralized) | Water (Aqua) | ad 100 |

TABLE 36
2-in-1 Shampoo

| Ingredients | INCI Name | Amount |
|---|---|---|
| Deionized water | Water | ad 100 |
| Shea butter | Butyrospermum Parkii (Shea) Butter | 0.1 |
| SymSave H | Hydroxyacetophenone | 0.5 |
| SymDiol 68 | 1.2 Hexanediol, Caprylyl Glycol | 0.5 |
| Plantacare PS 10 | Sodium Laureth Sulfate, Lauryl | 20.0 |
| | Glucoside | |
| Euperlan PK 771 | Glycol Distearate, Sodium Lauryl | 6.0 |
| | Sulfate, Cocamide MEA, Laureth-10 | |
| Sodium chloride | Sodium Chloride | 1.4 |
| Citric acid monohydrate | Citric acid | 0.1 |
| crystalline | | |
| Perfume oil PO1, PO2, | Fragrance | 0.5 |
| PO3, PO4, or PO5 | | |
| Zinc Omadine | Zinc pyrithione | 0.10 |
| 3-Hydroxypropyl | Propanediol Caprylate | 0.3 |
| caprylate | | |
| 3-Hydroxypropyl | Hydroxypropyl undecylenate | 0.15 |
| undecylenate | | |

TABLE 37
Body wash

| Ingredients | INCI | Amount |
|---|---|---|
| Lumerol K 28 | Disodium Laureth Sulfosuccinate, | 33.0 |
| | Cocamidopropyl Betaine, | |
| | Magnesium Lauryl Sulfate | |
| Amphotensid B 4 | Cocamidopropyl Betaine | 10.0 |
| Perlglanzmittel GM 4055 | MIPA-Pareth-25 Sulfate, Glycol | 4.0 |
| | Stearate | |
| Sodium Chloride | Sodium Chloride | 2.0 |
| Avocado oil | Persea Gratissima (Avocado) Oil | 3.0 |
| SymSave H | Hydroxyacetophenone | 0.8 |
| Hydrolite-5 Green | Pentylene Glycol | 1.0 |
| Water | Water | ad 100 |
| Perfume oil PO1, PO2, | Fragrance | 0.5 |
| PO3, PO4, or PO5 | | |
| 3-Hydroxypropyl | Propanediol Caprylate | 0.15 |
| caprylate | | |
| Glyceryl monocaprylate | Glyceryl caprylate | 0.15 |
| 3-Hydroxypropyl | Hydroxypropyl undecylenate | 0.1 |
| undecylenate | | |
| Glyceryl | Glyceryl undecylenate | 0.1 |
| monoundecylenate | | |

TABLE 38
Shower gel

| Ingredients | INCI | Amount |
|---|---|---|
| Deionized water | Water | ad 100 |
| Shea butter | Butyrospermum Parkii (Shea) | 1.0 |
| | Butter | |
| Plantacare PS 10 | Sodium Laureth Sulfate, Lauryl | 20.0 |
| | Glucoside | |
| Hydrolite-6 | 1.2 Hexanediol | 0.5 |
| Dehydroacetic acid | Dehydroacetic acid | 0.2 |
| SymSave H | Hydroxyacetophenone | 0.3 |
| Sodium chloride | Sodium Chloride | 1.4 |
| Citric acid monohydrate | Citric Acid | 1.3 |
| crystalline | | |
| Perfume oil PO1, PO2, | Fragrance | 0.6 |
| PO3, PO4, or PO5 | | |
| 3-Hydroxypropyl | Propanediol Caprylate | 0.1 |
| caprylate | | |
| Glyceryl | Glyceryl caprylate | 0.3 |
| monocaprylate | | |
| 3-Hydroxypropyl | Hydroxypropyl undecylenate | 0.2 |
| undecylenate | | |

TABLE 39
Intimate wash

| Ingredients | INCI | Amount |
|---|---|---|
| Tegobetaine HS | Cocamidopropyl Betaine, Glyceryl | 15.0 |
| | Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid. 80% | Lactic Acid | 0.1 |
| euxyl ® K700 | Potassium Sorbate, Benzyl Alcohol, | 0.3 |
| | Phenoxyethanol | |
| Water | Water | ad 100 |
| Perfume oil PO1, PO2, | Fragrance | 0.2 |
| PO3, PO4, or PO5 | | |
| 3-Hydroxypropyl | Propanediol Caprylate | 0.3 |
| caprylate | | |
| Hydrolite-5 Green | Pentylene Glycol | 0.3 |
| Glyceryl monocaprylate | Glyceryl caprylate | 0.1 |
| 3-Hydroxypropyl | Hydroxypropyl undecylenate | 0.1 |
| undecylenate | | |
| Glyceryl | Glyceryl undecylenate | 0.05 |
| monoundecylenate | | |

TABLE 40
Liquid soap. transparent

| Ingredients | INCI | Amount |
|---|---|---|
| Tagat O 2 | PEG-20 Glyceryl Oleate | 2.5 |
| Coconut oil diethanolamine | Cocamide DEA | 5.0 |
| condensate | | |
| Abil B 8842 | Cyclomethicone | 0.5 |
| Sodium laurylethersulfate, | Sodium Laureth Sulfate | 35.0 |
| 28% | | |
| Tego-Betaine L7 | Cocamidopropyl Betaine | 5.0 |
| Soap, 25% | Coconut acid, Potassium salt, | 20.0 |
| | Potassium Oleate | |
| Perfume oil PO1, PO2, | Fragrance | 0.4 |
| PO3, PO4, or PO5 | | |
| Preservative | DMDM Hydantoin | 0.2 |
| 3-Hydroxypropyl caprylate | Hydroxypropyl caprylate | 0.15 |
| Glyceryl monocaprylate | Glyceryl caprylate | 0.15 |
| Water | Water | ad 100 |

TABLE 41
Syndet soap. liquid

| Ingredients | INCI | Amount |
|---|---|---|
| Elfan OS 46 | Sodium Olefin C14-C16 Sulfonate | 35.5 |
| Armoteric LB | Lauryl Betaine | 8.0 |
| Euperlan PK 3000 OK | Glycol Distearate, Glycerin. Laureth- | 10.0 |
| | 4, Cocamidopropyl Betaine | |
| Elfacos GT 282 L | Talloweth-60 Myristyl Glycol | 3.0 |
| PCL-Liquid 100 | Cetearyl Ethylhexanoate | 4.0 |
| Perfume oil PO1, PO2, | Fragrance | 0.4 |
| PO3, PO4, or PO5 | | |
| SymSave H | 4-Hydroxyacetophenone | 0.6 |
| 3-Hydroxypropyl | Propanediol Caprylate | 0.15 |
| caprylate | | |
| 3-Hydroxypropyl | Hydroxypropyl undecylenate | 0.25 |
| undecylenate | | |
| Water | Water | ad 100 |

TABLE 42
Anti-acne wash

| Ingredients | Amount |
|---|---|
| Water (Aqua) | ad 100 |
| Polyquaternium-7 | 0.5 |
| Cocamidopropyl Betaine | 9.0 |
| Coco Glucoside | 2.0 |
| Polysorbate 80, Glycerol, Gossypium Herbaceum, | 1.0 |
| (Cotton) Seed Oil, Water (Aqua) | |
| Trideceth-9, PEG-5 Ethylhexanoate, Water | 1.0 |
| (Aqua) (Solubilizer) | |
| Glycereth-90 Isostearate, Laureth-2 | 0.5 |
| Sodium Laureth Sulfate | 37.0 |
| Glycerol, Triticum Vulgare (Wheat) Gluten, | 1.0 |
| Water (Aqua) (Dragoderm) | |
| Sodium Chloride | 0.3 |
| Perfume oil PO1, PO2, PO3, PO4, or PO5 | 1.0 |
| SymOcide BHO (Hydroxyacetophenone, Benzyl | 1.0 |
| alcohol, Caprylyl glycol, Water) | |
| 3-Hydroxypropyl caprylate | 0.25 |
| 3-Hydroxypropyl undecylenate | 0.15 |

TABLE 43
Mineral wash and cleaning gel

| Ingredients | INCI | Amount |
|---|---|---|
| Water | Water (Aqua) | ad 100 |
| Pionier ® NP 37 G | Sodium Carbomer | 1.5 |
| SymSol ® PF-3 | Water (Aqua), Pentylene Glycol, | 5.0 |
| | Sodium Lauryl Sulfoacetate, Sodium | |
| | Oleoyl Sarcosinate, Sodium Chloride, | |
| | Disodium Sulfoacetate, Sodium Oleate, | |
| | Sodium Sulfate | |
| Hydroviton ® 24 | Water (Aqua), Pentylene Glycol, | 1.0 |
| | Glycerol, Sodium Lactate, Lactic | |
| | Acid, Serine, Urea, Sorbitol, | |
| | Sodium Chloride, Allantoin | |
| Extrapone ® Silk GW | Water (Aqua), Glycerol, Hydrolyzed | 1.0 |
| | Silk | |
| Hydrolite ® 5 | Pentylene Glycol | 4.0 |
| Actipearls Red Star # | Water (Aqua), Propylene Glycol, | 1.0 |
| DH10402/6 | Algin, Gellan Gum, Xanthan Gum, | |
| | CalciumChloride, Cl 12490 (Pigment | |
| | Red 5), Mica (Cl 77019), Titanium | |
| | Dioxide (Cl 77891) | |
| Perfume oil PO1, PO2, | Fragrance | 0.5 |
| PO3, PO4, or PO5 | | |
| SymGuard CD | Phenylpropanol, o-cymen-5-ol, | 0.3 |
| | Decylene glycol | |
| 3-Hydroxypropyl | Propanediol Caprylate | 0.2 |
| caprylate | | |
| 3-Hydroxypropyl | Hydroxypropyl undecylenate | 0.1 |
| undecylenate | | |

TABLE 44
After Shave Tonic

| Ingredients | INCI | Amount |
|---|---|---|
| SymSol ® PF-3 | Water (Aqua), Pentylene Glycol, | 3.0 |
| | Sodium Lauryl Sulfoacetate, | |
| | Sodium Oleoyl Sarcosinate, | |
| | Sodium Chloride, Disodium | |
| | Sulfoacetate, SodiumOleate, | |
| | Sodium Sulfate | |
| SymSitive ® 1609 | Pentylene Glycol, 4-t- | 1.0 |
| | Butylcyclohexanol | |
| Frescolat ® ML | Menthyl Lactate | 0.3 |
| Glycerol 99.5 P. | Glycerol | 5.0 |
| Water | Water (Aqua) | ad 100 |
| Extrapone ® Glacier | Glycerol, Water (Aqua) | 1.0 |
| Water GW | | |
| SymCalmin ® | Butylene Glycol, Pentylene | 0.5 |
| | Glycol, Hydroxyphenyl | |
| | Propamidobenzoic Acid | |
| Dragosine ® | Carnosine | 0.1 |
| Hydrolite ® 5 | Pentylene Glycol | 5.0 |
| Ethanol 96% | Alcohol Denat. | 5.0 |
| Colour Pigment | Colour Pigment | 0.05 |
| Perfume oil PO1, PO2, | Fragrance | 0.15 |
| PO3, PO4, or PO5 | | |
| 3-Hydroxypropyl caprylate | Propanediol Caprylate | 0.1 |
| 3-Hydroxypropyl | Hydroxypropyl undecylenate | 0.05 |
| undecylenate | | |

TABLE 45
Hair conditioner with Crinipan. rinse-off

| Ingredients | INCI | Amount |
|---|---|---|
| Lanette ® O | Cetearyl Alcohol | 4.0 |
| Dragoxat 89 | Ethylhexyl Isononanoate | 2.0 |
| Genamin ® KDM-P | Behentrimonium Chloride | 1.0 |
| SymClariol | Decylene Glycol | 0.1 |
| SF 1550 | Phenyl Trimethicone | 0.1 |
| Neo Heliopan ® BB | Benzophenone-3 | 0.1 |
| Crinipan ® AD | Climbazole | 0.4 |
| Glycerol 99.5 P. | Glycerol | 6.0 |
| Water | Water (Aqua) | ad 100 |
| Actipone ® Alpha Pulp | Water (Aqua), Butylene Glycol, | 0.5 |
| | Malic Acid, Actinidia Chinensis | |
| | (Kiwi) Fruit Juice, Citrus | |
| | Aurantium Dulcis (Orange) Juice, | |
| | Citrus Paradisi (Grapefruit) Juice, | |
| | Pyrus Malus (Apple) Juice, | |
| | Trideceth-9, Prunus Amygdalus | |
| | Dulcis (Sweet Almond) Seed Extract | |
| Extrapone ® Bamboo P | Propylene Glycol, Water (Aqua), | 0.5 |
| | Butylene Glycol, Bambusa Vulgaris | |
| | Shoot Extract | |
| Sodium Hydroxide 10% | Sodium Hydroxide | 0.4 |
| aqueous solution | | |
| Colour I | Colour | 0.6 |
| Colour II | Colour | 0.3 |
| Perfume oil PO1, PO2, | Fragrance | 0.4 |
| PO3, PO4, or PO5 | | |
| Preservative | Methyl paraben | 0.3 |
| 3-Hydroxypropyl | Propanediol Caprylate | 0.15 |
| caprylate | | |
| Glyceryl monocaprylate | Glyceryl caprylate | 0.15 |
| 3-Hydroxypropyl | Hydroxypropyl undecylenate | 0.15 |
| undecylenate | | |

TABLE 46
Scalp soothing hair conditioner with
UV-B/UV-A protection. rinse off

| Ingredients | INCI | Amount |
|---|---|---|
| 3-Hydroxypropyl caprylate | Propanediol Caprylate | 0.2 |
| Glyceryl monocaprylate | Glyceryl caprylate | 0.2 |
| 3-Hydroxypropyl | Hydroxypropyl undecylenate | 0.1 |
| undecylenate | | |
| Glyceryl | Glyceryl undecylenate | 0.1 |
| monoundecylenate | | |
| Abil 350 | Dimethicone | 0.1 |
| Dehyquart A CA | Cetrimonium Chloride | 0.5 |
| Dehyquart SP | Quaternium-52 | 4.0 |
| Dracorin ® CE | Glyceryl Stearate Citrate | 1.0 |
| EDETA BD | Disodium EDTA | 0.1 |
| Extrapone ® Green Tea | Glycerin, Water (Aqua), | 0.7 |
| GW | Camellia Sinensis Leaf | |
| | Extract | |
| Perfume oil PO1, PO2, | Fragrance | 0.5 |
| PO3, PO4, or PO5 | | |
| Lara Care A-200 | Galactoarabinan | 0.5 |
| Neutral Oil | Caprylic/Capric Triglyceride | 1.0 |
| PCL Liquid 100 | Cetearyl Ethylhexoate | 0.3 |
| PCL Solid | Stearyl Heptanoate, Stearyl | 3.0 |
| | Caprylate | |
| SymOcide ®PS | Phenoxyethanol, Decylene Glycol, | 1.0 |
| | 1,2-Hexanediol | |
| Water (demineralized) | Water (Aqua) | ad 100 |

TABLE 47
Hair conditioner with UV protection

| Ingredients | INCI | Amount |
|---|---|---|
| Renex PEG 6000 | PEG-150 | 2.5 |
| Hair Conditioner Base | Cetyl alcohol, Behentrimonium | 3.0 |
| | chloride, Triticum Vulgare (Wheat) | |
| | bran extract, linoleic acid | |
| PCL-Solid | Stearyl heptanoate, stearyl | 0.5 |
| | caprylate | |
| Dow Corning 5200 | Laurylmethicone copolyol | 0.5 |
| Natrosol 250 HR | Hydroxyethylcellulose | 0.5 |
| Benzophenone-4 | Benzophenone-4 | 1.0 |
| Neo Heliopan AP | Disodiumphenyldibenz-imidazole | 1.0 |
| | tetrasulphonate | |
| Amino methyl propanol | Amino methyl propanol | 2.0 |
| Dow Corning 949 cationic | Amodimethicone, cetrimonium | 2.0 |
| emulsion | chloride, trideceth-12 | |
| Perfume oil PO1, PO2, | Fragrance | 0.8 |
| PO3, PO4, or PO5 | | |
| 1,2-Hexanediol | 1,2-Hexanediol | 0.5 |
| 3-Hydroxypropyl caprylate | Propanediol Caprylate | 0.2 |
| 3-Hydroxypropyl | Hydroxypropyl undecylenate | 0.1 |
| undecylenate | | |
| Water | Water (Aqua) | ad 100 |

TABLE 48
Hair conditioner. leave on

| Ingredients | INCI | Amount |
|---|---|---|
| 3-Hydroxypropyl caprylate | Propanediol Caprylate | 0.15 |
| Glyceryl monocaprylate | Glyceryl caprylate | 0.2 |
| 3-Hydroxypropyl | Hydroxypropyl undecylenate | 0.1 |
| undecylenate | | |
| Glyceryl | Glyceryl undecylenate | 0.05 |
| monoundecylenate | | |
| Dehyquart A CA | Cetrimonium Chloride | 0.2 |
| Dehyquart SP | Quaternium-52 | 2.0 |
| Dracorin ® CE | Glyceryl Stearate Citrate | 1.0 |
| Drago-Calm | Water, Glycerin, Avena Sativa | 2.0 |
| | (Oat) Kernel Extract | |
| Farnesol | Farnesol | |
| Perfume oil PO1, PO2, | Fragrance | 0.5 |
| PO3, PO4, or PO5 | | |
| Lara Care A-200 | Galactoarabinan | 0.1 |
| Polymer JR 400 | Polyquaternium-10 | 0.1 |
| Propylene Glycol | Propylene Glycol | 0.8 |
| SymMollient ® WS | Trideceth-9, PEG-5 Isononanoate, | 1.0 |
| | Water | |
| SymSol ®PF3 * | Water, Pentylene Glycol, Sodium | 1.5 |
| | Lauryl Sulfoacetate, Sodium | |
| | Oleoyl Sarcosinate, Sodium | |
| | Chloride, Disodium Sulfoacetate, | |
| | Sodium Oleate, Sodium Sulfate | |
| SymTriol ® | Caprylyl Glycol, 1,2-Hexanediol, | 1.0 |
| | Methylbenzyl Alcohol | |
| Water (demineralized) | Water (Aqua) | ad 100 |

TABLE 49
Anti-itch hair conditioner. leave on

| Ingredients | INCI | Amount |
|---|---|---|
| 3-Hydroxypropyl | Propanediol Caprylate | 0.15 |
| caprylate | | |
| Glyceryl monocaprylate | Glyceryl caprylate | 0.15 |
| 3-Hydroxypropyl | Hydroxypropyl undecylenate | 0.1 |
| undecylenate | | |
| (−)-alpha Bisabolol | Bisabolol | 0.1 |
| Dehyquart A CA | Cetrimonium Chloride | 0.5 |
| Dehyquart SP | Quaternium-52 | 4.0 |
| Dracorin ® CE* | Glyceryl Stearate Citrate | 1.0 |
| Drago-Oat-Active* | Water (Aqua), Butylene Glycol, | 2.0 |
| | Avena Sativa (Oat) Kernel | |
| | Extract | |
| Perfume oil PO1, PO2, | Fragrance | 0.1 |
| PO3, PO4, or PO5 | | |
| Lara Care A-200 | Galactoarabinan | 1.5 |
| Neutral Oil | Caprylic/Capric Triglyceride | 1.0 |
| PCL Liquid 100* | Cetearyl Ethylhexoate | 0.3 |
| Polymer JR 400 | Polyquaternium-10 | 0.1 |
| Propylene Glycol | Propylene Glycol | 0.8 |
| SymGlucan ® | Aqua, Glycerin, 1,2-Hexanediol, | 5 |
| | Caprylyl Glycol, Beta-Glucan | |
| SymMollient ® W/S | Trideceth-9, PEG-5 Isononanoate, | 2.0 |
| | Water (Aqua) | |
| SymOcide ® PH | Phenoxyethanol, | 1.2 |
| | Hydroxyacetophenone, Caprylyl | |
| | Glycol. Aqua | |
| SymSol ®PF3 * | Water, Pentylene Glycol, Sodium | 1.5 |
| | Lauryl Sulfoacetate, Sodium | |
| | Oleoyl Sarcosinate, Sodium | |
| | Chloride, Disodium Sulfoacetate, | |
| | Sodium Oleate, Sodium Sulfate | |
| Water, demineralized | Water (Aqua) | ad 100 |

TABLE 50
Sprayable hair conditioner with zinc pyrithrione. leave-on

| Ingredients | INCI | Amount |
|---|---|---|
| Monomuls 60-35 C | Hydrogenated Palm Glycerides | 1.7 |
| Cetiol OE | Dicaprylyl Ether | 7.2 |
| Abil 100 | Dimethicone | 3.6 |
| Dehyquart F 75 | Distearoylethyl | 4.0 |
| | Hydroxyethylmonium | |
| | Methosulfate, Cetearyl | |
| | Alcohol | |
| Eumulgin B1 | Ceteareth-12 | 3.5 |
| Cetiol S | Diethylhexylcyclohe xane | 7.2 |
| D-Panthenol | Panthenol | 0.1 |
| Glycerol 99.5 P. | Glycerol | 1.5 |
| Water | Water (Aqua) | ad 100 |
| Actipone ® Rosemary | Water (Aqua), Propylene Glycol, | 0.1 |
| | Rosmarinus Officinalis (Rosemary) | |
| | Leaf Extract | |
| Frescolat ® ML Cryst. | Menthyl Lactate | 0.5 |
| Dragosantol100 | Bisabolol | 0.1 |
| Zinc Omadine | Zinc pyrithione | 0.1 |
| Perfume oil PO1, PO2, | Fragrance | 0.4 |
| PO3, PO4, or PO5 | | |
| 2-Phenoxyethyl alcohol | Phenoxyethanol | 0.4 |
| SymSave H | Hydroxyacetophenone | 0.3 |
| SymDiol 68 | 1,2-Hexanediol, Caprylyl glycol | 0.3 |
| 3-Hydroxypropyl | Propanediol Caprylate | 0.4 |
| caprylate | | |
| 3-Hydroxypropyl | Hydroxypropyl undecylenate | 0.1 |
| undecylenate | | |

TABLE 51
Hair styling gel

| Ingredients | Amount |
|---|---|
| Water | ad 100 |
| PVM/MA Decadiene Crosspolymer | 0.6 |
| PVP | 3.0 |
| Isocetyl Stearate | 4.0 |
| Ethylhexyl Methoxycinnamate | 0.5 |
| Hydrolite-5 Green (Pentylene Glycol) | 0.5 |
| Aminomethyl Propanol | 0.4 |
| Perfume oil PO1, PO2, PO3, PO4, or PO5 | 0.6 |
| SymDiol ® 68T (1,2-Hexanediol, 1,2-Octanediol, Tropolone) | 0.4 |
| Phenoxyethanol | 0.3 |
| 3-Hydroxypropyl caprylate | 0.2 |
| 3-Hydroxypropyl undecylenate | 0.1 |

TABLE 52
Deodorant stick

| Ingredients | Amount |
|---|---|
| Sodium stearate | 8.0 |
| PPG-3 Myristyl ether | 70.0 |
| 1,2-propylene glycol | 10.0 |
| 1,1-dimethyl-3-phenylpropanol | 0.2 |
| 2-butyloctanoic acid | 0.2 |
| Perfume oil PO1, PO2, PO3, PO4, or PO5 | 0.6 |
| Water | ad 100 |
| SymDeo Plus | 0.5 |
| (Jasmol (2-benzylheptanol), 1-Dodecanol (Lauryl Alcohol), 1,2- | |
| Decanediol (Decylene Glycol), 2-Phenoxyethyl Alcohol | |
| (Phenoxyethanol)) | |
| 3-Hydroxypropyl caprylate | 0.15 |
| Glyceryl monocaprylate | 0.15 |
| 3-Hydroxypropyl undecylenate | 0.1 |

TABLE 53
Zirconium suspensoid antiperspirant stick

| Ingredients | INCI | Amount |
|---|---|---|
| PCL Liquid 100 | Cetearyl ethylhexanonate | ad 100 |
| Silicone Fluid 345 | Cyclomethicone | 10.0 |
| CRODACOL C90 | Cetyl Alcohol | 8.0 |
| SYNCROWAX HGLC | C18-36 Triglyceride | 8.0 |
| CRODAMOL PTC | Pentaerythritol | 5.0 |
| | Tetracaprylate/Caprate | |
| SymDeo MPP | Dimethyl Phenylbutanol | 0.3 |
| SYNCROWAX HRC | Tribehenin | 4.0 |
| VOLPO N5 | Oleth-5 | 1.0 |
| Titanium Dioxide | | 1.0 |
| Rezal 36GP | Aluminium Tetrachlorohydrex | 20.0 |
| | GLY | |
| Dry Flo C | Aluminium Starch Octenyl | 22.5 |
| | Succinate | |
| Preservative | Phenoxyethanol | 0.8 |
| Perfume oil PO1, PO2, | Fragrance | 0.6 |
| PO3, PO4, or PO5 | | |
| 3-Hydroxypropyl | Propanediol Caprylate | 0.15 |
| caprylate | | |
| Glyceryl monocaprylate | Glyceryl caprylate | 0.15 |
| 3-Hydroxypropyl | Hydroxypropyl undecylenate | 0.1 |
| undecylenate | | |
| Glyceryl monoundecylenate | Glyceryl undecylenate | 0.1 |

TABLE 54
Antiperspirant/deodorant roll-on

| Ingredients | INCI | Amount |
|---|---|---|
| 3-Hydroxypropyl caprylate | Propanediol Caprylate | 0.15 |
| Glyceryl monocaprylate | Glyceryl caprylate | 0.15 |
| 3-Hydroxypropyl | Hydroxypropyl undecylenate | 0.1 |
| undecylenate | | |
| Dragosantol ® 100* | Bisabolol | 0.1 |
| Ethanol 96% | Ethanol | 30.0 |
| Farnesol | Farnesol | 0.5 |
| Perfume oil PO1, PO2, | Fragrance | 1.5 |
| PO3, PO4, or PO5 | | |
| Frescolat ®ML cryst. | Menthyl Lactate | 0.2 |
| Irgasan DP 300 | Triclosan | 0.3 |
| Natrosol 250 HHR | Hydroxyethyl-cellulose | 0.3 |
| Solubilizer 611674 | PEG-40 Hydrogenated Castor Oil, | 2.0 |
| | Trideceth-9, Water (Aqua) | |
| SymDeo ® B125 | 2-Methyl 5-Cyclohexylpentanol | 0.5 |
| Water (demineralized) | Water (Aqua) | ad 100 |
| Zirkonal L 450 | Aluminium Zirconium | 37.0 |
| | Pentachlorohydrate | |
| | (40% aqueous solution) | |

TABLE 55
Deodorant formulation in the form of a roll-on gel

| Ingredients | Amount |
|---|---|
| 1.3-butylene glycol | 2.0 |
| 2-Methyl 5-cyclohexylpentanol | 0.1 |
| PEG-40-hydrogenated castor oil | 2.0 |
| Hydroxyethylcellulose | 0.5 |
| Pentylene Glycol | 1.0 |
| Perfume oil PO1, PO2, PO3, PO4, or PO5 | 0.3 |
| 1,3-propanediol | 0.5 |
| SymGuard CD (3-Phenylpropanol, o-cymen-3-ol, | 0.4 |
| Decylene glycol) | |
| Ethylhexyl glycerin | 0.1 |
| 3-Hydroxypropyl caprylate | 0.3 |
| Glyceryl monocaprylate | 0.15 |
| 3-Hydroxypropyl undecylenate | 0.1 |
| Water | ad 100 |

TABLE 56
Clear deo anti-perspirant roll-on

| Ingredients | INCI | Amount |
|---|---|---|
| Methocel E4M Premium | Hydroxypropyl Methylcellulose | 0.5 |
| Water | Water (Aqua) | ad 100 |
| Neo-PCL Water Soluble | Trideceth-9, PEG-5 | 1.0 |
| N | Ethylhexanoate, Water (Aqua) | |
| Solubilizer | PEG-40 Hydrogenated Castor Oil, | 3.0 |
| | Trideceth-9, Propylene Glycol, | |
| | Water (Aqua) | |
| Deolite | Dimethyl Phenylpropanol, | 0.5 |
| | Pentylene Glycol | |
| Locron LW | Aluminium Chlorohydrate | 25.0 |
| Aloe Vera Gel | Aloe Barbadensis Leaf Juice | 1.0 |
| Concentrate 10/1 | | |
| 1,2-Propylene Glycol 99 P | Propylene Glycol | 4.0 |
| GC | | |
| Ethanol 96% | Alcohol Denat. | 30.0 |
| Perfume oil PO1, PO2, | Fragrance | 1.0 |
| PO3, PO4, or PO5 | | |
| 3-Hydroxypropyl caprylate | Propanediol Caprylate | 0.3 |
| 3-Hydroxypropyl | Hydroxypropyl undecylenate | 0.15 |
| undecylenate | | |

TABLE 57
Deodorant pump spray with SymClariol

| Ingredients | INCI | Amount |
|---|---|---|
| SymClariol ® | Decylene Glycol | 0.2 |
| Solubilizer | PEG-40 Hydrogenated Castor Oil, | 4.0 |
| | Trideceth-9, Propylene Glycol, | |
| | Water (Aqua) | |
| Neo-PCL Water Soluble | Trideceth-9, PEG-5 | 1.5 |
| N | Ethylhexanoate, Aqua | |
| SymRelief ® | Bisabolol, Zingiber Officinale | 0.1 |
| | (Ginger) Root Extract | |
| Water | Water (Aqua) | ad 100 |
| 1,2-Propylene Glycol | Propylene Glycol | 6.0 |
| Perfume oil PO1, PO2, | Perfume | 0.4 |
| PO3, PO4, or PO5 | | |
| SymDiol ® 68 | 1,2-Hexanediol, Caprylyl Glycol | 0.2 |
| 3-Hydroxypropyl caprylate | Propanediol Caprylate | 0.2 |
| 3-Hydroxypropyl | Hydroxypropyl undecylenate | 0.1 |
| undecylenate | | |

TABLE 58
Whitening deodorant spray

| Ingredients | Amount |
|---|---|
| PEG-40-hydrogenated castor oil | 3.0 |
| Ethylhexylglycerol (Octoxyglycerol) | 0.2 |
| Symbright 2036 (Sclareolide) | 0.1 |
| Ethanol | 40.0 |
| Citrate buffer | 0.5 |
| 1,2-Hexanediol. 1,2-Octanediol (1:1) | 0.3 |
| SymOcide C (o-cymen-5-ol) | 0.05 |
| 2-Benzylheptan-1-ol (Jasmol) | 0.1 |
| Perfume oil PO1, PO2, PO3, PO4, or PO5 | 0.75 |
| Phenoxyethanol | 0.4 |
| 3-Hydroxypropyl caprylate | 0.2 |
| Glyceryl monoundecylenate | 0.1 |
| Water | ad 100 |

TABLE 59
Deodorant Aoerosol Spray

| Ingredients | Amount |
|---|---|
| 3-Hydroxypropyl caprylate | 0.2 |
| Disiloxane | Ad 100 |
| Isoadipate | 5.0 |
| C12-C15 Alkyl Benzoate | 10.0 |
| Tocopheryl Acetate | 0.5 |
| Farnesol | 0.3 |

40% bulk charged with 60% Propane/Butane

TABLE 60
Sunscreen lotion (o/w, broadband protection)

| Ingredients | INCI | Amount |
|---|---|---|
| 3-Hydroxypropyl caprylate | Propanediol Caprylate | 0.15 |
| Glyceryl monocaprylate | Glyceryl caprylate | 0.15 |
| 3-Hydroxypropyl | Hydroxypropyl undecylenate | 0.1 |
| undecylenate | | |
| Glyceryl monoundecylenate | Glyceryl undecylenate | 0.1 |
| Carbopol Ultrez-10 | Carbomer | 0.2 |
| Dow Corning 246 Fluid | Cyclohexasiloxane and | 2.0 |
| | Cyclopentasiloxane | |
| Dragosantol ® 100* | Bisabolol | 0.3 |
| EDETA BD | Disodium EDTA | 0.1 |
| Emulsiphos ® | Potassium Cetyl Phosphate, | 1.5 |
| | Hydrogenated Palm Glycerides | |
| Perfume oil PO1, PO2, | Fragrance | 0.4 |
| PO3, PO4, or PO5 | | |
| Frescolat ®MGA | Menthone Glycerol Acetal | 0.3 |
| Glycerol 85% | Glycerin | 4.7 |
| Keltrol RD | Xanthan Gum | 0.2 |
| Lanette O | Cetearyl Alcohol | 1.0 |
| Neo Heliopan ® 357 | Butyl Methoxy-dibenzoyl-methane | 1.0 |
| Neo Heliopan ® AP | Disodium Phenyl Dibenzimidazole | 10.0 |
| (10% as sodium salt) | Tetrasulfonate | |
| Neo Heliopan ® AV | Ethylhexyl Methoxy-cinnamate | 3.0 |
| Neo Heliopan ® Hydro | Phenylbenz-imidazole Sulfonic | 6.7 |
| (15% as sodium salt) | Acid | |
| Neo Heliopan ® MBC | 4-Methylbenzyl-idene Camphor | 1.5 |
| Neo Heliopan ® OS | Ethyl hexyl Salicylate | 5.0 |
| Neutral Oil | Caprylic/Capric Triglyceride | 2.0 |
| SymMatrix ® | Maltodextrin. Rubus Fruticosus | 0.3 |
| | (Blackberry) Leaf Extract | |
| SymOcide ® BHO | Hydroxyacetophenone, Benzyl | 1.5 |
| | alcohol, Caprylyl Glycol, Aqua | |
| Tegosoft TN | C12-15 Alkyl Benzoate | 5.0 |
| Tocopherol Acetate | Tocopheryl Acetate | 0.5 |
| Triethanolamine. 99% | Triethanolamine | 0.5 |
| Water (demineralized) | Water (Aqua) | ad 100 |

TABLE 61
Emulsion with UV-A/B-broadband protection

| Ingredients | INCI | Amount |
|---|---|---|
| 3-Hydroxypropyl caprylate | Propanediol Caprylate | 0.1 |
| Glyceryl monocaprylate | Glyceryl caprylate | 0.1 |
| 3-Hydroxypropyl | Hydroxypropyl undecylenate | 0.05 |
| undecylenate | | |
| Glyceryl monoundecylenate | Glyceryl undecylenate | 0.05 |
| Abil 350 | Dimethicone | 0.3 |
| Butylene Glycol | Butylene Glycol | 3.0 |
| Carbopol Ultrez-10 | Carbomer | 0.2 |
| Citric Acid 10% sol. | Citric Acid | 0.3 |
| Dragosantol ® 100* | Bisabolol | 0.1 |
| EDETA BD | Disodium EDTA | 0.1 |
| Emulsiphos ® | Potassium Cetyl Phosphate, | 1.5 |
| | Hydrogenated Palm Glycerides | |
| Perfume oil PO1, PO2, | Fragrance | 0.1 |
| PO3, PO4, or PO5 | | |
| Frescolat ®X-COOL | Menthyl Ethylamido Oxalate | 1.0 |
| Glyceryl Stearate | Glyceryl Stearate | 2.0 |
| Keltrol RD | Xanthan Gum | 0.2 |
| Lanette 16 | Cetyl Alcohol | 1.2 |
| Lanette E | Sodium Cetearyl Sulfate | 0.7 |
| Neo Heliopan ® AP (10% as | Disodium Phenyl | 22.0 |
| sodium salt) | Dibenzimidazole Tetrasulfonate | |
| Neo Heliopan ® HMS | Homosalate | 5.0 |
| Neutral Oil | Caprylic/Capric Triglyceride | 2.0 |
| PCL Liquid 100 | Cetearyl Ethylhexoate | 3.0 |
| Sodium Hydroxide (10% | Sodium Hydroxide | 2.8 |
| sol.) | | |
| Symdiol ®68 | 1,2-Hexanediol, Caprylylglycol | 0.5 |
| SymMollient ®S | Cetearyl Nonanoate | 1.5 |
| SymSitive ® 1609 | Pentylene Glycol, 4-t- | 0.5 |
| | Butylcyclohexanol | |
| SymWhite ®377 | Phenylethyl Resorcinol | 0.5 |
| Tocopherol Acetate | Tocopheryl Acetate | 0.5 |
| Water (demineralized) | Water (Aqua) | ad 100 |

TABLE 62
Sun protection soft cream (w/o). SPF 40

| Ingredients | INCI | Amount |
|---|---|---|
| Dehymuls PGPH | Polyglyceryl-2 dipolyhydroxy- | 5.0 |
| | stearate | |
| Copherol 1250 | Tocopheryl acetate | 0.5 |
| Permulgin 3220 | Ozocerite | 0.5 |
| Zinc stearate | Zinc stearate | 0.5 |
| Tegosoft TN | C12-15 Alkyl benzoate | 10.0 |
| Neo Heliopan ® E1000 | Isoamyl-p-methoxycinnamate | 2.0 |
| Neo Heliopan ® 303 | Octocrylene | 5.0 |
| Neo Heliopan ® MBC | 4-Methylbenzylidene camphor | 3.0 |
| Zinc oxide, neutral | Zinc oxide | 5.0 |
| Water, distilled | Water (aqua) | ad 100 |
| EDETA BD | Disodium EDTA | 0.1 |
| Glycerol | Glycerol | 4.0 |
| Magnesium sulfate | Magnesium sulfate | 0.5 |
| Perfume oil PO1, PO2, | Fragrance | 0.3 |
| PO3, PO4, or PO5 | | |
| Symdiol ® 68 | 1,2-Hexanediol, Caprylylglycol | 0.3 |
| 3-Hydroxypropyl | Propanediol Caprylate | 0.15 |
| caprylate | | |
| Glyceryl monocaprylate | Glyceryl caprylate | 0.15 |

TABLE 63
Sun protection milk (w/o)

| Ingredients | INCI | Amount |
|---|---|---|
| Dehymuls PGPH | Polyglyceryl-2 dipolyhydroxy- | 3.0 |
| | stearate | |
| Beeswax 8100 | Beeswax | 1.0 |
| Monomuls 90-0-18 | Glyceryl oleate | 1.0 |
| Zinc stearate | Zinc stearate | 1.0 |
| Hydrolite-8 | Caprylyl Glycol | 0.3 |
| Cetiol SN | Cetearyl isononanoate | 5.0 |
| Cetiol OE | Dicaprylyl ether | 5.0 |
| Tegosoft TN | C12-15 alkyl benzoate | 4.0 |
| Vitamin E | Tocopherol | 0.5 |
| Neo Heliopan ® OS | Ethylhexyl salicylate | 5.0 |
| Neo Heliopan ® AV | Ethylhexyl methoxycinnamate | 7.5 |
| Uvinul ® T150 | Ethylhexyl triazone | 1.5 |
| Water. distilled | Water (Aqua) | ad 100 |
| Trilon BD | Disodium EDTA | 0.1 |
| Glycerol | Glycerol | 5.0 |
| Neo Heliopan ® AP | Disodium phenyl dibenzimidazole | 15.0 |
| 10% solution, neutralized | tetrasulfonate | |
| with NaOH | | |
| Perfume oil PO1, PO2, | Fragrance | 0.25 |
| PO3, PO4, or PO5 | | |
| Alpha bisabolol | Bisabolol | 0.1 |
| SymOcide ® PT | Phenoxyethanol, Tropolone | 0.25 |
| 3-Hydroxypropyl caprylate | Propanediol Caprylate | 0.2 |
| 3-Hydroxypropyl | Hydroxypropyl undecylenate | 0.1 |
| undecylenate | | |

TABLE 64
Sun spray with UV-A/B-broadband protection with low oil content

| Ingredients | INCI | Amount |
|---|---|---|
| 3-Hydroxypropyl | Propanediol Caprylate | 0.15 |
| caprylate | | |
| Glyceryl monocaprylate | Glyceryl caprylate | 0.15 |
| 3-Hydroxypropyl | Hydroxypropyl undecylenate | 0.05 |
| undecylenate | | |
| Glyceryl monoundecylenate | Glyceryl undecylenate | 0.05 |
| Ethanol 96% | Ethanol | 13.0 |
| Perfume oil PO1, PO2, | Fragrance | 0.5 |
| PO3, PO4, or PO5 | | |
| Glyceryl Stearate | Glyceryl Stearate | 4.0 |
| Hydroviton ® PLUS | Water, Pentylene Glycol, Glycerin, | 1.0 |
| | Fructose, Urea, Citric Acid, | |
| | Sodium Hydroxide, Maltose, | |
| | Sodium PCA, Sodium Chloride, | |
| | Sodium Lactate, Trehalose, | |
| | Allantoin, Sodium hyaluronate, | |
| | Glucose | |
| Isoadipate ® | Diisopropyl Adipate | 1.0 |
| Neo Heliopan ® AV | Ethylhexyl Methoxy-cinnamate | 25.0 |
| Neo Heliopan ® MBC | 4-Methylbenzyl-idene Camphor | 33.3 |
| Propylene Glycol | Propylene Glycol | 0.8 |
| Tego Betain L7 | Cocamidopropyl Betaine | 1.0 |
| Water (demineralized) | Water (Aqua) | ad 100 |

TABLE 65
Sunscreen spray o/w, SPE 15-20

| Ingredients | INCI | Amount |
|---|---|---|
| Dracorin ® GOC | Glyceryl Oleate Citrate, Caprylic/ | 2.0 |
| | Capric Triglyceride | |
| Corapan ® TQ | Diethylhexyl 2,6-Naphthalate | 3.0 |
| Neo Heliopan ® HMS | Homosalate | 7.0 |
| Neo Heliopan ® OS | Ethylhexyl Salicylate | 5.0 |
| Neo Heliopan ® 357 | Butyl Methoxydibenzoylmethane | 3.0 |
| Isoadipate | Diisopropyl Adipate | 6.0 |
| Baysilone ® Oil M10 | Dimethicone | 1.0 |
| Edeta ® BD | Disodium EDTA | 0.1 |
| Vitamin E Acetate | Tocopheryl Acetate | 0.5 |
| Dragosantol ® 100 | Bisabolol | 0.1 |
| Pemulen ® TR-2 | Acrylates/C10-30 Alkyl Acrylate | 0.25 |
| | Crosspolymer | |
| Water | Water (Aqua) | ad 100 |
| Glycerol 99.5 P. | Glycerol | 4.0 |
| Butylene Glycol | Butylene Glycol | 5.0 |
| Neo Heliopan ® Hydro | Phenylbenzimidazole Sulfonic | 8.0 |
| (103089). used as 25% | Acid | |
| aq. Solution, | | |
| neutralized with Biotive ® | | |
| L-Arginine | | |
| Biotive ® L-Arginine | Arginine | 0.55 |
| Perfume oil PO1, PO2, | Fragrance | 0.4 |
| PO3, PO4, or PO5 | | |
| SymOcide PS | Phenoxyethanol, 1,2-Hexanediol, | 0.8 |
| | Decylene glycol | |
| 3-Hydroxypropyl caprylate | Propanediol Caprylate | 0.2 |
| 3-Hydroxypropyl | Hydroxypropyl undecylenate | 0.1 |
| undecylenate | | |

TABLE 66
After sun gel

| Ingredients | INCI | Amount |
|---|---|---|
| SymSol ® PF-3 | Water (Aqua), Pentylene Glycol, | 3.0 |
| | Sodium Lauryl Sulfoacetate, | |
| | Sodium Oleoyl Sarcosinate, Sodium | |
| | Chloride, Disodium Sulfoacetate, | |
| | Sodium Oleate, Sodium Sulfate | |
| Glycerol 99.5 P. | Glycerol | 5.0 |
| SymHelios ® 1031 | Benzylidene Dimethoxydimethylin | 0.1 |
| | danone | |
| Water | Water (Aqua) | ad 100 |
| Pemulen ® TR-2 | Acrylates/C10-30 Alkyl Acrylate | 1.0 |
| | Crosspolymer | |
| D-Panthenol 75 W | Panthenol | 0.5 |
| SymFinity ® 1298 | Echinacea Purpurea Extract | 0.1 |
| Extrapone ® Pearl GW | Water (Aqua), Glycerol, Hydrolyzed | 1.0 |
| | Pearl, Xanthan Gum | |
| Sodium Hydroxide 10% | Sodium Hydroxide | 2.5 |
| solution | | |
| Ethanol 96% | Alcohol Denat. | 15.0 |
| Perfume oil PO1, PO2, | Fragrance | 0.2 |
| PO3, PO4, or PO5 | | |
| SymOcide ® PS | Phenoxyethanol, 1,2-Hexanediol, | 0.8 |
| | Decyleneglycol | |
| 3-Hydroxypropyl | Propanediol Caprylate | 0.15 |
| caprylate | | |
| 3-Hydroxypropyl | Hydroxypropyl undecylenate | 0.05 |
| undecylenate | | |

TABLE 67
After sun lotion

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| Perfume oil PO1, PO2, PO3, PO4, or PO5 | 0.30 |
| 1,2-Hexanediol | 1.0 |
| 4-Hydroxyacetophenone | 0.3 |
| Pentylene glycol | 4.0 |
| Aqua dem. | ad 100 |
| Triethanolamine | 0.2 |
| 3-Hydroxypropyl caprylate | 0.2 |
| 3-Hydroxypropyl undecylenate | 0.1 |

TABLE 68
Syndet antimicrobial soap bar

| Ingredients | INCI | Amount |
|---|---|---|
| Zetesap 813 A | Disodium Lauryl Sulfosuccinate, | ad 100 |
| | Sodium Lauryl Sulfate, Corn | |
| | Starch, Cetearyl Alcohol, | |
| | Paraffin, Titanium Dioxide | |
| Amphotensid GB 2009 | Disodium Cocoamphodiacetate | 6.0 |
| Allantoin | Allantoin | 1.0 |
| Perfume oil PO1, PO2, | Fragrance | 1.0 |
| PO3, PO4, or PO5 | | |
| SymOcide C | o-cymen-5-ol | 0.1 |
| 3-Hydroxypropyl | Propanediol Caprylate | 0.1 |
| caprylate | | |
| Glyceryl monocaprylate | Glyceryl caprylate | 0.1 |
| 3-Hydroxypropyl | Hydroxypropyl undecylenate | 0.05 |
| undecylenate | | |
| Glyceryl monoundecylenate | Glyceryl undecylenate | 0.05 |

TABLE 69
Syndet soap bar

| Ingredients | INCI | Amount |
|---|---|---|
| Fenopon AC-78 | Sodium Cocoyl Isethionate | 20.0 |
| Natriumlaurylsulfoacetate | Sodium Lauryl Sulfoacetate | 16.0 |
| Paraffin | Paraffin | 19.0 |
| Wax. microcrystalline | Microcrystalline Wax | 1.0 |
| Corn Starch | Corn Starch | 8.0 |
| Coconut acid | Coconut acid | 2.0 |
| Lauric acid diethanol amide | Lauramide DEA | 2.0 |
| Dextrin | Dextrin | 21.0 |
| Lactic acid. 88% | Lactic Acid | 1.0 |
| SymGuard CD | 3-Phenylpropanol, o-cymen-5-ol, | 0.3 |
| | Decylene glycol | |
| Thymol | Thymol | 0.05 |
| Water | Water | ad 100 |
| Perfume oil PO1, PO2, | Fragrance | 1.0 |
| PO3, PO4, or PO5 | | |
| 3-Hydroxypropyl caprylate | Propanediol Caprylate | 0.15 |
| Glyceryl monocaprylate | Glyceryl caprylate | 0.15 |
| 3-Hydroxypropyl | Hydroxypropyl undecylenate | 0.05 |
| undecylenate | | |
| Glyceryl monoundecylenate | Glyceryl undecylenate | 0.05 |

TABLE 70
Antimicrobial toilet soap bar

| Ingredients | Amount |
|---|---|
| Sodium soap from tallow | 60.0 |
| Sodium soap from palm oil | 27.0 |
| Pentylene Glycol | 0.5 |
| Glycerol | 2.0 |
| Sodium Chloride | 0.5 |
| 1-Hydroxyethane-1,1-diphosphonicacid, tetrasodium salt | 0.3 |
| Alpha-Tocopherol | 0.1 |
| Pigment Yellow 1 | 0.02 |
| Water | ad 100 |
| Perfume oil PO1, PO2, PO3, PO4, or PO5 | 3.0 |
| Farnesol | 0.2 |
| 3-Hydroxypropyl caprylate | 0.1 |
| Glyceryl monocaprylate | 0.1 |
| 3-Hydroxypropyl undecylenate | 0.05 |
| Glyceryl monoundecylenate | 0.05 |

TABLE 71
Shaving foam

| Ingredients | Amount |
|---|---|
| Dem. Water | ad 100 |
| Triethanolamine | 4.0 |
| Edenor L2 SM (Stearinic acid, Palmitinic acid) | 5.3 |
| Laureth-23 | 3.0 |
| Stearylalcohol | 0.5 |
| SymOcide BHO | 1.0 |
| (Hydroxacetophenone, Benzyl alcohol, Caprylyl Glycol, Water) | |
| 3-Hydroxypropyl caprylate | 0.2 |
| Glyceryl monocaprylate | 0.1 |
| 3-Hydroxypropyl undecylenate | 0.1 |
| Glyceryl monoundecylenate | 0.05 |
| Sodium lauryl sulfate | 3.0 |
| Extrapone Seaweed (water, propylene glycol, potassium iodide, | 1.0 |
| Fucus Vesiculosus Extract) | |
| Dragosantol (Bisabolol) | 0.1 |
| Perfume oil PO1, PO2, PO3, PO4, or PO5 | 1.0 |
| Propane, butane 4.2 bar | 4.0 |

TABLE 72
Sprayable disinfecting gel

| Ingredients | INCI | Amount |
|---|---|---|
| Water | Water (Aqua) | ad 100 |
| Stabileze QM | PVM/Ma Decadiene | 0.25 |
| | Crosspolymer | |
| Sodium Hydroxide 10% | Sodium Hydroxide | 0.4 |
| solution | | |
| Coffein pure | Caffeine | 0.5 |
| Extrapone ® Horse | Propylene Glycol, Water (Aqua), | 1.0 |
| Chestnut | Glucose, Aesculus Hippocastanum | |
| | (Horse Chestnut) Seed Extract, | |
| | Lactic Acid | |
| Hydrolite ® 5 | Pentylene Glycol | 3.0 |
| Xylityl Sesquicaprylate | Xylityl Sesquicaprylate | |
| 1.3 Butylene Glycol | Butylene Glycol | 5.0 |
| Biotive ® Esculin | Esculin | 0.3 |
| Sesquihydrate | | |
| Ethanol 96% | Alcohol Denat. | 10.0 |
| Solubilizer | PEG-40 Hydrogenated Castor Oil, | 0.5 |
| | Trideceth-9, Water (Aqua) | |
| Perfume oil PO1, PO2, | Fragrance | 0.2 |
| PO3, PO4, or PO5 | | |
| Octenidine dihydrochloride | Octenidine dihydrochloride | 0.1 |
| Preservative | Phenoxyethanol | 0.7 |
| 3-Hydroxypropyl caprylate | Propanediol Caprylate | 0.15 |
| Glyceryl monocaprylate | Glyceryl caprylate | 0.15 |
| 3-Hydroxypropyl | Hydroxypropyl undecylenate | 0.05 |
| undecylenate | | |
| Glyceryl monoundecylenate | Glyceryl undecylenate | 0.05 |

TABLE 73
Solution for wet wipes

| Ingredients | INCI | Amount |
|---|---|---|
| SymSol ® PF-3 | Water (Aqua), Pentylene Glycol, | 2.0 |
| | Sodium Lauryl Sulfoacetate, | |
| | SodiumOleoyl Sarcosinate, Sodium | |
| | Chloride, Disodium Sulfoacetate, | |
| | SodiumOleate, Sodium Sulfate | |
| Dragosantol ® 100 | Bisabolol | 0.1 |
| Glycerol 99.5 P. | Glycerol | 5.0 |
| Water | Water (Aqua) | ad 100 |
| Hydrolite ® 5 | Pentylene Glycol | 5.0 |
| D-Panthenol 75 W | Panthenol | 0.8 |
| DragoCalm ® | Water (Aqua), Glycerol, Avena | 1.0 |
| | Sativa (Oat) Kernel Extract | |
| Witch Hazel-Distillate | Hamamelis Virginiana (Witch Hazel) | 1.0 |
| | Water, Water (Aqua), Alcohol | |
| Allplant Essence ® Org. | Pelargonium Graveolens | 1.0 |
| Rose Geranium P | Flower/Leaf/Stem Water | |
| Perfume oil PO1, PO2, | Fragrance | 0.1 |
| PO3, PO4, or PO5 | | |
| SymOcide BHO | Benzyl Alcohol, | 0.8 |
| | Hydroxyacetophenone, Caprylyl | |
| | glycol, Water | |
| 3-Hydroxypropyl | Propanediol Caprylate | 0.2 |
| caprylate | | |
| Glyceryl monocaprylate | Glyceryl caprylate | 0.2 |
| 3-Hydroxypropyl | Hydroxypropyl undecylenate | 0.1 |
| undecylenate | | |
| Glyceryl | Glyceryl undecylenate | 0.1 |
| monoundecylenate | | |

1-15. (canceled)

16. A method for preventing and/or reducing growth of microorganisms comprising incorporating one or more fatty acid esters chosen from 3-hydroxypropyl caprylate, 3-hydroxypropyl undecylenate, and glyceryl monoundecylenate into a cosmetic product, wherein incorporation of the one or more fatty acid esters:
(i) inhibits growth of microorganisms on a mammal's skin or mucosa upon application of the product to the mammal's skin or mucosa; and/or
(ii) preserves the product against microbial growth.

17. The method of claim 16 comprising incorporation of:
two or more of the fatty acid esters, wherein at least one of the two or more fatty esters is chosen from 3-hydroxypropyl caprylate, 3-hydroxypropyl undecylenate, and glyceryl monoundecylenate;
two or more fatty acid esters, wherein at least one of the two or more fatty esters is chosen from 3-hydroxypropyl caprylate and 3-hydroxypropyl undecylenate; or
one or more fatty acid esters, wherein one of the one or more fatty esters is hydroxypropyl caprylate.

18. The method of claim 16, wherein the one or more fatty acid esters:
(iii) modify physicochemical properties of the product,
(iv) improve sensory properties of the product when applied to the mammal's skin or mucosa, and/or
(v) balance and/or reduce sebum reduction when the product is applied to the mammal's skin or mucosa.

19. The method according to claim 16, wherein the microorganisms are natural microbiota of mammalian skin or mucosa.

20. The method according to claim 16, wherein the microorganisms are chosen from the genus Staphylococcus, Corynebacterium, Aspergillus, Candida, Escherichia, Peptoniphilus, Streptococcus, Lactobacillus, Gardnerella, Fannyhessea, Epidermophyton, Trichophyton, Streptococcus, Fusobacterium, and mixtures thereof.

21. The method according to claim 20, wherein the microorganisms are chosen from Staphylococcus epidermidis, Staphylococcus hominis, Corynebacterium xerosis, Aspergillus brasiliensis, Candida albicans, Escherichia coli, Staphylococcus aureus, Peptoniphilus lacrimalis, Streptococcus agalactiae, Lactobacillus acidophilus, Gardnerella vaginalis, Fannyhessea vaginae (Atopobium vaginae), Epidermophyton floccosum, Trichophyton rubrum, Streptococcus mutans, Fusobacterium nucleatum, and mixtures thereof.

22. The method according to claim 16, wherein the product is chosen from body deodorant care products, skin care and cleansing products, intimate care and cleansing products, foot care and cleansing products, and oral care products.

23. The method according to claim 16, wherein the product further comprises one or more 1,2-alkane diol(s).

24. The method according to claim 23, wherein the one or more 1,2-alkane diol(s) are chosen from 1,2-pentanediol, 1,2-hexanediol, 1,2-octanediol, and 1,2-decanediol, and mixtures thereof.

25. The method according to claim 16, wherein the product further comprises one or more compounds chosen from dimethyl phenylbutanol, glyceryl caprylate, and Farnesol.

26. The method according to claim 16, wherein the product comprises two or more fatty acid esters chosen from:
3-hydroxypropyl caprylate and 3-hydroxypropyl undecylenate, or
glyceryl monocaprylate and 3-hydroxypropyl undecylenate, or
3-hydroxypropyl caprylate and glyceryl monoundecylenate, or
glyceryl monocaprylate and glyceryl monoundecylenate, or
3-hydroxypropyl caprylate and glyceryl monocaprylate.

27. A cosmetic product comprising:
one or more fatty acid esters chosen from 3-hydroxypropyl caprylate, 3-hydroxypropyl undecylenate, and glyceryl monoundecylenate;
two or more fatty acid esters chosen from 3-hydroxypropyl caprylate, 3-hydroxypropyl undecylenate, and glyceryl monoundecylenate; or
3-hydroxypropyl caprylate and 3-hydroxypropyl undecylenate; or
one or more fatty acid esters, wherein one of the one or more fatty esters is hydroxypropyl caprylate;
wherein the product provides antimicrobial activity upon application to a mammal's skin or mucosa.

28. The product according to claim 27, wherein the antimicrobial activity is sufficient for treating a condition or infection of the skin or mucosa involving an excess of microorganisms in the region of the skin or mucosa.

29. The product according to claim 28, wherein the microorganisms are natural microbiota of mammalian skin or mucosa,

30. The product according to claim 29, wherein the microorganisms are chosen from the genus Staphylococcus, Corynebacterium, Aspergillus, Candida, Escherichia, Peptoniphilus, Streptococcus, Lactobacillus, Gardnerella, Fannyhessea, Epidermophyton, Trichophyton, Streptococcus, Fusobacterium, and mixtures threreof.

31. The product according to claim 29, wherein the microorganisms are chosen from epidermidis, Staphylococcus hominis, Corynebacterium xerosis, Aspergillus brasiliensis, Candida albicans, Escherichia coli, Staphylococcus aureus, Peptoniphilus lacrimalis, Streptococcus agalactiae, Lactobacillus acidophilus, Gardnerella vaginalis, Fannyhessea vaginae (Atopobium vaginae), Epidermophyton floccosum, Trichophyton rubrum, Streptococcus mutans, Fusobacterium nucleatum, and mixtures threreof.

32. The product of claim 27 chosen from body deodorant care products, skin care and cleansing products, intimate care cleansing products, foot care and cleansing products, and oral care products.

33. A cosmetic product comprising:
one or more fatty acid esters chosen from 3-hydroxypropyl caprylate, 3-hydroxypropyl undecylenate, and glyceryl monoundecylenate;
two or more fatty acid esters chosen from 3-hydroxypropyl caprylate, 3-hydroxypropyl undecylenate, and glyceryl monoundecylenate;
two or more fatty acid esters, wherein at least one of the two or more fatty esters is chosen from 3-hydroxypropyl caprylate and 3-hydroxypropyl undecylenate; or
one or more fatty acid esters, wherein one of the one or more fatty esters is hydroxypropyl caprylate.

34. The product of claim 33, wherein the one or more fatty acid esters is in an amount:
(i) sufficient for inhibiting growth of microorganisms on a mammal's skin or mucosa upon application of the product to the mammal's skin or mucosa;
(ii) sufficient for preserving the cosmetic product against microbial growth.

35. The product of claim 33 chosen from body deodorant care products, skin care and cleansing products, intimate care cleansing products, foot care and cleansing products, and oral care products.
